(19) United States
(12) Patent Application Publication
(10) Pub. No.: US 2026/0354848 A1
(43) Pub. Date: Nov. 12, 2026

(54) APPARATUS AND METHODS FOR FLUID CLASSIFICATION AND DYNAMIC CONTROL IN ASPIRATION SYSTEMS

(71) Applicant: J. Michael SHIFFLETTE, Alachua, FL (US)

(72) Inventor: J. Michael SHIFFLETTE, Alachua, FL (US)

(21) Appl. No.: 19/677,702

(22) Filed: May 14, 2026

Related U.S. Application Data
(63) Continuation of application No. 17/409,635, filed on Aug. 23, 2021.
(60) Provisional application No. 63/069,224, filed on Aug. 24, 2020.
(60) Provisional application No. 63/143,230, filed on Jan. 29, 2021.

(51) Int. Cl. A61B 17/22; A61B 17/3203; A61B 90/00

(52) U.S. Cl. CPC A61B 17/22; A61B 17/32037; A61B 90/06; A61B 2017/22079; A61B 2090/064; A61B 2217/005

(57) ABSTRACT

An apparatus and method for fluid classification and dynamic control in an aspiration system includes a conduit, a controlled source of differential pressure (CSDP) fluidically coupled to the conduit, a sensor configured to measure pressure within the conduit, and a controller operatively coupled to the CSDP and the sensor. The controller determines a pressure measurement corresponding to operation of the CSDP at a setpoint and determines a data sub-range corresponding to the pressure measurement. Operation of the aspiration system is updated based upon the data sub-range. In some embodiments, the controller selects or updates an operating mode based upon the data sub-range, the operating mode comprising one or more control parameters associated with operation of the aspiration system.

### STATEMENT OF RELATED CASES

This case is a continuation of U.S. patent application Ser. No. 17/409,635, filed Aug. 23, 2021, entitled “MULTI-MODE VISCOMETRIC THROMBECTOMY SYSTEM,” the entire disclosure of which is incorporated by reference herein.

### FIELD OF THE INVENTION

The present invention relates generally to aspiration systems and, more particularly, to apparatus and methods for real-time fluid classification and dynamic control in aspiration systems.

### BACKGROUND OF THE INVENTION

Thrombectomy procedures are intended to dislodge and subsequently aspirate thrombus extracorporeally. Many current thrombectomy systems employ vacuum/suction or hydrodynamic pressure gradients which extract both thrombus and blood concurrently from the patient. Suction thrombectomy systems employ a pressure gradient between the vacuum source and the catheter tip to dislodge and extract thrombus. Such a system is limited to a maximum differential pressure of less than about 15 psi (<30inHg vacuum). This pressure gradient over the diameter/length parameters of the catheter will cause water, blood and thrombus to flow at significantly different rates, in decreasing order. A catheter filled with water will flow at a greater rate than the same catheter filled with blood because of the difference in viscosity. Thrombus-laden blood flows more slowly than blood due to of an increase in viscosity (and particle size) which vary with the concentration and composition of thrombus present.

Ideally, the thrombectomy catheter tip is positioned immediately adjacent to thrombus and thereby draws in predominantly thrombus for extraction. But if the thrombectomy catheter tip is positioned even a short distance away from thrombus, then viable blood will be preferentially aspirated due to blood's comparatively low viscosity. Low-viscosity blood flows around viscous, coagulated thrombus, which results in blood loss without effective thrombus extraction. Therefore, the ratio of viable blood to thrombus is often sub-optimal which results in clinical complications (including exsanguination and limitations on procedure times). A thrombectomy catheter that is deployed in thrombus-free blood will flow viable blood directly into a waste container. When in the vicinity of thrombus, both viable blood and thrombus are extracted concurrently at rates and proportions that are not under system detection and control; both viable blood and thrombus are collected in a single waste container. For any given differential pressure across a catheter, viable blood flows at a greater rate than thrombus-laden blood; the proportions of viable blood to thrombus in the waste container are sub-optimal because of the differing flow rates of different aspirate compositions. The improved thrombectomy procedure will extract greater amounts of thrombus and lesser amounts of blood from the patient.

Suction thrombectomy systems are also subject to clogging because certain thrombus compositions become lodged in the catheter. The clog may be a coagulation/coalescence of smaller thrombi, or a single clump of thrombus that is too large to traverse the catheter length, given the pressure gradient available. Large thrombi are preferentially sought during thrombectomy procedures because extracting these results in improved patient outcome within a given procedure duration. However, the process of clearing a clog in a catheter can require additional components and techniques that require time and expertise. The slow process of clearing a clog thereby limits the overall procedure efficacy because of time constraints. An improved thrombectomy system, with the ability to clear large thrombi initially (without the time delay of manual clog-clearing), would afford more procedure time available to be dedicated to more numerous, smaller thrombi as well, thus improving procedure efficacy.

Suction thrombectomy systems exhibit two detrimental characteristics: indiscriminately aspirating viable blood and thrombus at rates inversely proportional to procedure efficacy, and a propensity for the catheter to clog which results in delays to the procedure and limits the number of sites than can be addressed in a single procedure.

Measuring the viscosity of the aspirate is a rational approach to determining the thrombus concentration of the aspirate; the system could thereby selectively aspirate thrombus at a high flow rate and viable blood at a low flow rate. But viscometry is typically a batch process (e.g., Brookfield viscometer), wherein a rotating cylinder is immersed in the liquid to be analyzed. Batch viscometry is not feasible for use in a thrombectomy system because the time delay between sample collection and analysis is too long for any control system response. Alternatively, continuous flow/process viscometers are commercially available, but they are expensive and require a dedicated control system that would require integration to the thrombectomy control system. The component cost and complexity of integrating a process viscometer into a thrombectomy system are strong detractors to such an approach.

### SUMMARY OF THE INVENTION

In the description that follows, a number of terms are utilized. In order to provide a clear and consistent understanding of the specification and claims, including the scope to be given such terms, the following definitions are provided.
Viscometer—An instrument that measures the viscosity of fluids. Herein, viscometer also means any apparatus that employs any system of creating variable flow through a catheter and that concurrently measures the flowing fluid pressure within the catheter and subsequently determines the viscosity of the fluid contained within the catheter. Viscosity is measured in arbitrary units which may or may not be converted to engineering units, e.g., oil exhibits 40 pressure units at 50% differential pressure.
Differential Viscometer—An instrument that measures the difference in viscosity between two or more liquids, e.g., water and oil, or an unknown liquid and reference liquid. Differential viscometry may be expressed in relative units. Example: water exhibits 10 pressure units at 50% differential pressure: the viscosity of water is 25% of the viscosity of oil at 50% differential pressure.
Catheter—any fluid conduit with a large length-to-diameter aspect ratio greater than approximately 50. Diameters may range from sub-millimeter to meters (at corresponding lengths). For flow calculation example purposes, herein a representative catheter is considered to be in the ranges of 3 Fr to 12 Fr (1 mm to 4 mm) diameter and between 50 cm and 200 cm in length.
Viscosity—the resistance of a fluid to flow; herein including the resistance of a homogeneous liquid or inhomogeneous mixture of liquids and/or solids to flow through a catheter. Example: an inhomogeneous mixture of thrombus and blood which may be uniformly distributed or spatially discrete along the length of a catheter. The viscosity of this inhomogeneous mixture may be measured by a viscometer.
Aspirate (noun)—any liquid, solid, slurry or heterogeneous matter transferred through a catheter; also the contents of the catheter.
Aspirate (verb)—employ pressure, vacuum, pump or any system to transfer any liquid, solid, slurry or heterogeneous matter through a catheter.
Aspiration or Positive Aspiration—The net removal of fluid from any reservoir including the patient vascular system; resultant from any number of inflow cycles; net mass transfer is into the catheter.
Neutral or Isovolumetric Aspiration—Aspiration with concurrent infusion such that there is negligible net mass transfer into or out of the catheter. Also called a neutral or isovolumetric cycle.
Negative aspiration—Infusion of extracorporeal liquid at a rate that exceeds the aspiration rate. Resultant from any number of outflow cycles; net mass transfer is out of the catheter.
Aspirate Characteristic or Fluid Characteristic—Attribute classification of aspirate or any fluid into subsets by any logical means, including statistical inference or other algorithm. Example aspirate characteristics include: blood, saline, thrombus, SAE 30 motor oil, SAE 0W40 motor oil, clot and clog, etc. Notation used herein may include aspirate characteristic=blood, (aspirate characteristic=thrombus), aspirate characteristic=clog, (fluid characteristic=SAE 30 motor oil), etc.
Controlled source of differential pressure—any setpoint controlled system that causes fluid to flow within a catheter. Examples include the shaft speed of a pump (0%, 10%, 20%, 30% . . . 100% of full speed) or an evacuated reservoir operating at variable vacuum level (0 mmHg, 10 mmHg, 20 mmHg, 25 mmHg), etc.
Aspirate Pump—a liquid pump that is in fluid communication with a reservoir through a catheter lumen. Capable of generating a differential pressure that causes fluid flow in either direction through the catheter. Example: setpoint-controlled peristaltic pump, capable of suction head exceeding 20 inHg. Rotational speed range is approximately 6 RPM to 3000 RPM (0.1 Hz to 50 Hz).
Infusion Pump—a liquid pump that is in fluid communication with a reservoir through a catheter lumen. Example: setpoint-controlled piston pump, capable of pressures ranging from 3 psi to 10,000 psi. Rotational speed range is approximately 6 RPM to 3000 RPM (0.1 Hz to 50 Hz).
Setpoint—The desired value of a control output, e.g., pump speed, vacuum, pressure or temperature. Herein also, the analog or digital output, from system controller, that changes the magnitude of the controlled output.
Update—The act of refreshing a control output (e.g., setpoint, thrombectomy operating mode, aspirate characteristic) to either a new or unchanged value. Control outputs are updated periodically and not necessarily simultaneous with any other event.
Aspiration Setpoint—A setpoint of the controlled source of differential pressure; at least 2 positive aspiration setpoints exist. A positive aspiration setpoint results in flow in the aspiration flow direction; a negative aspiration setpoint (reverses the aspirate pump shaft rotational direction) results in flow in the infusion direction.
Infusion Setpoint—A setpoint of infusion pump speed; at least 3 infusion setpoints exist (including 0, or off).
Valve Setpoint—A setpoint of the position of a valve. Only two states exist for an on-off valve: open and closed.
Thrombus—Any coalescence of blood components which remains attached to the vascular system. Herein, thrombus also includes mobile emboli (detached thrombi) as a result of any phenomena including a thrombectomy procedure. Mobile emboli, aspirated by catheter are herein also considered thrombus or thrombi.
Pump Inlet Pressure—Fluid pressure measured in the vicinity of a pump inlet, herein typically vacuum in range of 0 to 29+ in Hg; also the analog or digital output of a pressure transducer located near the pump inlet.
Reservoir—Any fluid source or sink, including infinite and finite. Examples include the atmosphere, an ocean, a bottle, a syringe, or intravascular blood (the patient bloodstream is a reservoir).
Aperture Direction—The outward normal direction of an aperture (hole) in any surface; herein applying to the orientation of a hole in a catheter for mass transfer.
Pump Cycle—A single 360 degree rotation of the shaft of a pump. Typically on the order of 1 second duration; range of 0.030 seconds to 10 seconds. Pump cycles may be repeated for all integer and non-integer cycle counts, e.g., 1.10 cycles herein describes a 396 degree rotation of the shaft.
Inflow Cycle—a pump cycle of aspirate pump wherein aspirate flow rate exceeds infusion flow rate for a net inflow from a patient or any reservoir.
Outflow Cycle—a pump cycle of infusion pump wherein infusion flow rate exceeds aspiration flow rate for a net outflow into a patient or any reservoir.
Thrombectomy Operating Mode—Any mode of operating a thrombectomy system comprising (independent and simultaneous) setpoint control of at least one system that effects characteristic flow regimes (e.g., viscometric inflow sampling, thrombus extraction, positive/neutral/negative aspiration, hydrodynamic thrombus maceration, radial direct impingement, clog clearing, etc.) both internal and external to the catheter including the vicinity of the catheter aperture. A thrombectomy operating mode may be changed within a procedure by the occurrence of any event, including: pump cycle count, data from analytical instrumentation, lapse of time, operator input, etc. Example: any number of infusion outflow cycles may precede any number of aspiration inflow cycles followed by any number of viscometric inflow sampling cycles. A thrombectomy procedure thereby comprises a finite number of pump cycles; at an average of 1 cycle per second, a 15 minute procedure comprises approximately 900 pump cycles. A thrombectomy operating mode is comprised of any number of infusion and/or aspirate pump cycles which may be identical and repeated or updated and changed on a cycle-to-cycle basis. A thrombectomy operating mode may therefore be updated more than 100 times during any single thrombectomy procedure.
Data Set—A matrix of measured value vs. aspiration setpoint for any fluid in any catheter. Example: aspirate pump inlet pressure (dependent variable) vs. aspirate pump speed (independent variable). Because aspirate pump inlet pressure is a function of aspirate pump speed, a (2-dimensional) data set is appropriate for pressure. Other analytical instrumentation systems, e.g., conductivity and absorbance, are not strong functions of aspirate pump speed. For such analytical instrumentation systems, a single scalar value is generally valid across a range of aspirate pump speeds; in this case, the matrix is one-dimensional.
Homogeneous data—data, e.g., viscometric, from measurements of a single liquid sample, e.g., water. The physical properties of the liquid sample do not change during the time that data are collected.
Inhomogeneous data—data, e.g., viscometric, from measurements of a time-dependent array of different liquids, e.g., water, blood, thrombus, SAE30 motor oil, etc.
Sub-Range—A range of inhomogeneous data that has been divided into sub-ranges that are assigned fluid characteristics, e.g., water, blood, thrombus, SAE30 motor oil, etc. The fluid characteristic of an unknown liquid is thereby determined.
SPC—Statistical Process Control.
Control Chart—SPC technique to determine process changes over time.
UCL, LCL—Upper Control Limit and Lower Control Limits of control charts.

Disclosed is a thrombectomy system that automates a thrombectomy procedure to: (1) minimize procedure time, (2) minimize loss of viable patient blood, (3) permit more thorough and complete thrombus extraction and (4) reduce the required clinician attentiveness and skill level. Several novel subsystems are individually disclosed; these subsystems are integrated into the thrombectomy system of the present invention. The subsystems may be employed individually or collectively and are germane to applications more far-reaching than thrombectomy or medicine; environmental, industrial, automotive and other applications are encompassed.

The thrombectomy system of the present invention comprises one or more of the following subsystems:
(1) Differential viscometer to instantaneously and quantitatively determine the viscous aspirate characteristic of the aspirate contained within the catheter.
(2) Discrete analytical instrumentation systems (light absorption, conductivity, etc.) to instantaneously and quantitatively determine additional aspirate characteristics (within the catheter) at the location of the analytical instrumentation.
(3) A plurality of reservoirs for the collection of aspirate: a reservoir for viable blood (for reinfusion) and a second reservoir for thrombus-laden waste.
(4) A saline infusion subsystem to clear clogs, flush the catheter of thrombotic debris and provide hydrodynamic ablation/maceration of thrombus.
(5) A variable aperture catheter that facilitates the clearing of clogs and also provides aspiration and thrombus extraction selectively in radial or axial directions.
(6) A control system that collects and analyzes (including statistically) aspirate data and that updates a setpoint of a controlled source of differential pressure multiple times within a single thrombectomy procedure. Control system also provides incremental clinician feedback for efficient device positioning and thrombectomy procedure endpoint determination.

Disclosed is a thrombectomy system that comprises a variable-speed liquid aspirate pump, a catheter, and a pressure transducer (typically disposed near the aspirate pump inlet); these three components form a viscometer by which aspirate viscosity is measured and aspirate characteristic is determined by system controller. The thrombectomy system thereby identifies and discriminates between viable and thrombus-laden blood, whereby the different aspirate components are diverted to separate collection reservoirs, with viable blood segregated from thrombus and available to be filtered and reinfused, as indicated by clinical conditions.

Disclosed is a thrombectomy system that comprises an infusion pump and an aspirate pump independently responding to multiple changes in setpoint during the course of a single thrombectomy procedure; the infusion pump and aspirate pump collectively provide positive/neutral/negative aspiration, depending upon clinical data including aspirate characteristic. Operation of the aspirate pump while the infusion pump is off results in net inflow to the catheter, or positive aspiration. Operation of the infusion pump while the aspirate pump is off results in net outflow from the catheter, or negative aspiration. Concurrent operation of the infusion pump and the aspirate pump will allow the aforementioned positive or negative aspiration, but also neutral (isovolumetric) aspiration, depending upon the selected aspiration setpoint and infusion setpoint. Any temporal combination of these operational flow regimes is exploited by the thrombectomy system of the present invention resulting in improved clinical efficiency and efficacy. The infusion pump and aspirate pump also act in concert to clear clogs, modify flow rates/pressure gradients and macerate stubborn/wall-adherent thrombus. Adjustable apertures in the catheter permit axial or radial thrombus maceration and extraction.

Disclosed is real-time detection of aspirate characteristics, including: saline, blood, thrombus, clot, clog, etc., by employing analytical instrumentation in the aspirate stream. Viscometry, absorption of electromagnetic radiation, e.g., light, or measurement of aspirate electrical conductivity are but three examples of such analytical instrumentation systems. The use of SPC control chart techniques is employed (as an example) to accurately and immediately detect a change of aspirate characteristic in the aspirate stream. Aspirate characterized as viable blood (aspirate characteristic=blood) may hence be aspirated at a minimum flow rate and collected for filtration and reinfusion; at other times within a single thrombectomy procedure, aspirate characterized as thrombus laden (aspirate characteristic=thrombus) may be rapidly aspirated and diverted to waste.

Disclosed is a thrombectomy catheter comprising one or more variable apertures that modify local flowfield velocities and permit many combinations of radial and axial inflow. Axial inflow is appropriate for centralized venous/arterial occlusions; radial inflow is appropriate for softer wall-adherent thrombus. Direct impingement radial outflow is appropriate for more stubborn wall-adherent thrombus.

Disclosed is a thrombectomy control system that concurrently and independently controls any or all of:
(1) aspirate pump setpoint.
(2) infusion pump setpoint,
(3) variable area catheter actuator setpoint, and
(4) setpoint control of valve network to divert aspirate to/from a plurality of reservoirs.

The thrombectomy control system utilizes input data including, but not limited to:
(1) viscometric aspirate variable data,
(2) photometric/absorption/conductivity variable data, and
(3) clinician input.

Variable data are identified for SPC analysis of a (flowing liquid) process stream (subject to analyses such as viscometric and photometric); this permits rapid detection of any change in aspirate characteristic under ever-changing clinical conditions.

### BRIEF DESCRIPTION OF THE DRAWINGS

FIG. 1A shows a block diagram of the thrombectomy system of the present invention.

FIG. 1B shows example thrombectomy operating modes which may be executed during a single thrombectomy procedure.

FIG. 1C shows a representative flowchart for the thrombectomy system of the FIG. 1A.

FIG. 2 shows a block diagram of a catheter-based differential viscometer that detects the viscosity of aspirate contained within the length of the catheter.

FIG. 3 shows aspirate pump inlet pressure vs aspirate pump speed in tabular and graphical forms.

FIG. 4A graphs viscometry data for common liquids in arbitrary units. The reference pressure is atmospheric pressure.

FIG. 4B graphs differential viscometry data for common liquids normalized to a reference liquid (water) by subtraction.

FIG. 4C shows example aspirate pump inlet pressure at 5 pump speeds in tabular form. The data range is divided into sub-ranges corresponding to the fluid characteristic.

FIG. 5A shows an oblique view of an embodiment of a probe viscometer.

FIG. 5B shows a cutaway view of probe viscometer.

FIG. 6 shows a cutaway view of conductivity meter.

FIG. 7 shows an oblique view of photo-detector.

FIG. 8A shows a cutaway view of photo-detector instantaneously measuring the light absorption of blood in the catheter at the vicinity of the photo-detector.

FIG. 8B shows a SPC control chart tracking the % transmittance (absorbance) of viable blood vs time.

FIG. 9A shows a cutaway view of photo-detector instantaneously measuring the light absorption of thrombus in the catheter at the vicinity of the photo-detector. Viable blood is flowing toward the photo-detector and is to be subsequently analyzed.

FIG. 9B shows a SPC control chart tracking the % transmittance (absorbance) of thrombus vs time.

FIG. 10A shows a partial cutaway view of a generalized catheter (prior art) comprised of a second lumen for the infusion of saline under pressure to emanate from axial, tangential and radial orifices into the catheter tip region.

FIG. 10B shows an end-view of the generalized catheter of FIG. 10A (prior art). Radial and tangential flows are depicted by arrows emanating from the respective orifices.

FIG. 10C is a block diagram depicting the liquid communication pathways for the generalized catheter of FIG. 10A. Disclosed is the inclusion of analytical instrumentation and a system controller.

FIG. 11A shows the clog clearing tip in section view; the aspirate is blood and saline is in hydrodynamic tubing.

FIG. 11B shows the clog clearing tip in section view; thrombus is shown clogging the distal tip. Blood is downstream of thrombus.

FIG. 11C shows the clog clearing tip in section view; thrombus is shown partially eroded and saline is downstream of thrombus.

FIG. 12A shows an oblique view of variable aperture tip in closed configuration; inflow is axial through the distal tip. Radial jet impacts the catheter.

FIG. 12B shows an oblique view of variable aperture tip in a second configuration that exposes a radial aperture and increases the distance from the radial jet to the distal tip.

FIG. 12C shows an oblique view of variable aperture tip in a third configuration with open radial aperture such that radial jet impinges upon surrounding tissue. Inflow is predominantly radial.

FIG. 12D shows an oblique view of variable aperture tip in a fourth configuration with fully open radial aperture and fully retracted sheath.

FIG. 13A shows an oblique view of variable aperture tip with elastic tip shown deployed; elastic tip restricts the axial inflow to produce substantially radial inflow.

FIG. 13B shows an oblique view of variable aperture tip with elastic tip shown stowed; elastic tip exposes both axial and radial apertures and obstructs radial hydrodynamic jet (not show for clarity).

### DETAILED DESCRIPTION OF THE DRAWINGS

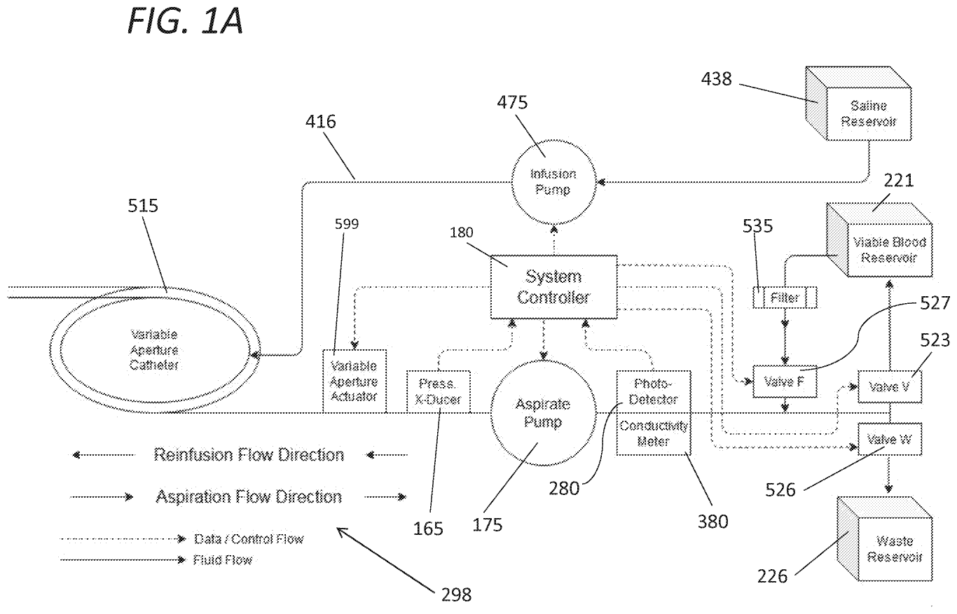

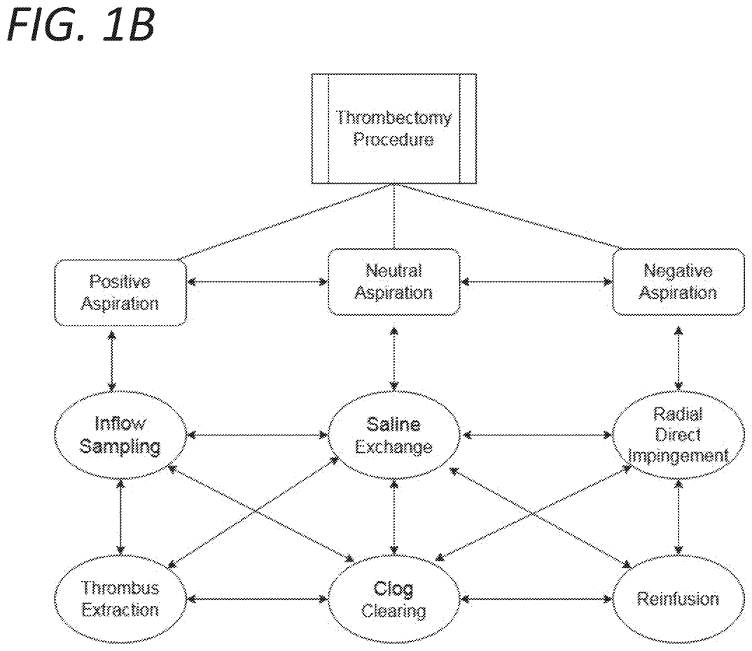

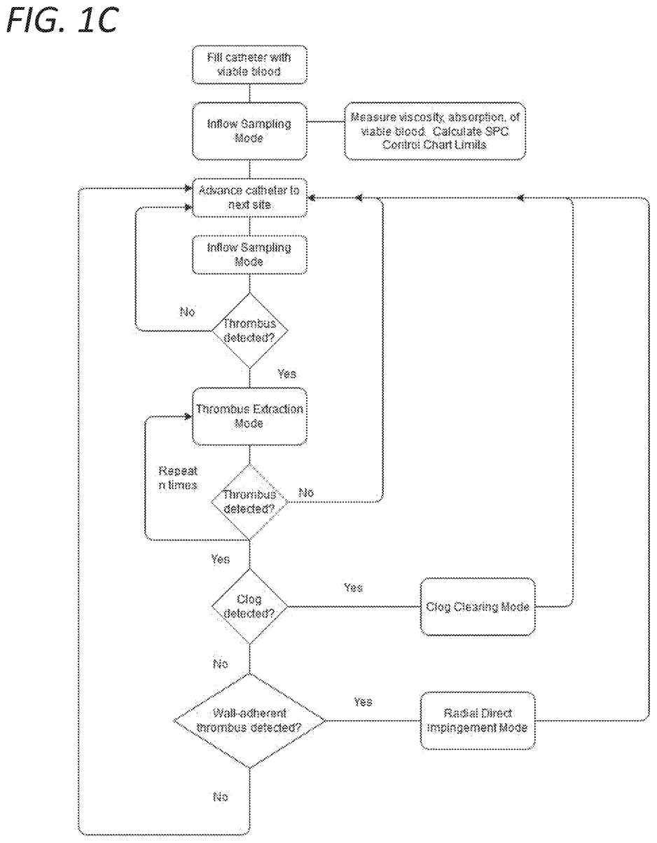

FIG. 1A is a block diagram of the thrombectomy system of the present invention. A system controller 180 receives data from analytical instrumentation comprising pressure transducer 165, photo-detector 280, and conductivity meter 380; system controller 180 subsequently determines the aspirate characteristic. Concomitantly, aspirate pump 175 and infusion pump 475 receive independent setpoints that are updated by system controller 180 (in response to updated aspirate characteristics) during the course of a single thrombectomy procedure. System controller 180 concurrently controls the setpoints of valve W 526, valve V 523, and valve F 527 to establish desired fluid communication pathways between variable aperture catheter 515, waste reservoir 226, and viable blood reservoir 221.

The thrombectomy system of FIG. 1A eclipses prior art because system controller 180 determines the aspirate characteristic and responds with a coordinated sequence of actions to improve both the efficiency and efficacy of a thrombectomy procedure. Each example aspirate characteristic thereby evokes an appropriate example control response: viable blood is slowly aspirated and diverted to viable blood reservoir 221, thrombus-laden blood is rapidly aspirated and diverted to waste reservoir 226, a clog in variable aperture catheter 515 is cleared by setpoint coordination of aspirate pump 175 and infusion pump 475. This gives rise to the implementation of multiple thrombectomy operating modes that are executed sequentially within a single thrombectomy procedure.

FIG. 1B illustrates how a thrombectomy procedure is performed by sequentially executing a plurality of thrombectomy operating modes. The thrombectomy operating modes are classified as positive, neutral and negative aspiration modes. FIG. 1B shows example thrombectomy operating modes which are sequentially executed by system controller 180 based upon aspirate characterization data from analytical instrumentation 450. Example positive aspiration modes include inflow sampling and thrombus extraction. Example neutral aspiration modes include saline exchange and clog clearing. Example negative aspiration modes include radial direct impingement and reinfusion. FIG. 1C illustrates how a single thrombectomy procedure is subdivided into a plurality of thrombectomy operating modes; each operating mode consisting of a finite number of pump cycles. In some thrombectomy operating modes, e.g., radial direct impingement and clog clearing, dissimilar pump cycles are sequentially executed to perform a specific function within that mode. Conversely, the inflow sampling mode may consist of repeated, similar pump cycles which are sequentially executed to perform the function of ongoing aspirate characterization. FIG. 1B illustrates that any thrombectomy operating mode may be preceded or followed by any other thrombectomy operating mode. Subdividing a single thrombectomy procedure into the sequential execution of multiple thrombectomy operating modes is an inventive of the present invention.

A first example thrombectomy operating mode is inflow sampling mode wherein: aspirate pump 175 induces aspirate flow in variable aperture catheter 515 in the aspiration flow direction 298, infusion pump 475 is generally off. Inflow sampling mode is typically employed whenever the aspirate characteristic is viable blood, system controller 180 outputs valve setpoints of valve W 526 to off, valve V 523 to on, and valve F 527 to off; thus diverting aspirate to viable blood reservoir 221. Any number of cycles of aspirate pump 175 may occur without a change in aspirate characteristic; system controller 180 or the clinician may infer that the variable aperture catheter 515 is not optimally positioned for thrombus extraction. System controller 180 provides incremental clinician feedback through sounds incrementally varying in pitch and volume; slight changes in variable data from analytical instrumentation are thereby conveyed to the clinician.

A second example thrombectomy operating mode is thrombus extraction mode wherein: system controller 180 outputs valve setpoints of valve W 526 to on, valve V 523 to off, and valve F 527 to off; thus diverting aspirate to waste reservoir 226. System controller 180 increases aspiration setpoint for aspirate pump 175 to increase aspirate flow rate for rapid thrombus extraction; system controller 180 concurrently monitors for any change in aspirate characteristic.

A third example thrombectomy operating mode is clog clearing mode wherein: system controller 180 outputs valve setpoints of valve W 526 to on, valve V 523 to off, and valve F 527 to off; thus diverting aspirate to waste. System controller 180 subsequently and independently controls the aspiration setpoint and the infusion setpoint to enact any of the following example flow regimes:

Exchange of saline for aspirate in variable aperture catheter 515 to reduce aspirate viscosity; accomplished by coordinating aspirate pump 175 setpoint and infusion pump 475 setpoint for generally neutral aspiration and without significant mass transfer across a catheter aperture.

Aspiration setpoint and/or infusion setpoint repeatedly or alternatingly changed from negative to positive, to dislodge clogged thrombus akin to rocking a stuck car. Comprised of alternating inflow and outflow cycles.

Increased infusion setpoint to employ hydrodynamic maceration effects within variable aperture catheter 515 to clear the clog. Comprised of any sequential combination of inflow, neutral and outflow cycles.

A fourth example thrombectomy operating mode is radial direct impingement mode wherein: system controller 180 outputs valve setpoints of valve W 526 to on, valve V 523 to off, and valve F 527 to off; thus diverting aspirate to waste. System controller 180 subsequently and independently controls the aspiration setpoint, the infusion setpoint, and the variable aperture actuator 599 setpoint. Variable aperture catheter 515 is configured to open radial aperture 580; this permits both aspiration and infusion to occur in the radial direction of variable aperture catheter 515. Infusion setpoint is controlled (for any number of consecutive infusion pump cycles) to a value that results in direct impingement of radial jet 424 upon surrounding tissue, e.g., thrombus, clot, vessel wall, or blood. The velocity of radial jet 424 is a function of infusion setpoint and therefore system controller 180 controls the destructive power of radial jet 424 upon direct impingent of surrounding tissue. Concurrently, aspiration setpoint may be controlled to any positive value, though net outflow cycles are preferred. The duration of direct impingement of radial jet 424 upon surrounding tissue is a function of the number of infusion pump 475 cycles and is approximately 10 cycles or fewer and approximately 1 second duration. Direct impingement of radial jet 424 upon surrounding tissue is enacted by system controller 180 to macerate, erode and/or dislodge thrombus, including wall-adherent thrombus. A short duration of direct impingement of radial jet 424 upon surrounding tissue is optimal because only a small quantity of thrombotic debris are created with each infusion pump cycle. Interspersing thrombus extraction inflow cycles with direct impingement outflow cycles optimizes efficient thrombus removal because only a small quantity of thrombotic debris is released during direct impingement outflow cycles to be aspirated in subsequent thrombus extraction cycles.

A fifth example thrombectomy operating mode is reinfusion outflow mode wherein: system controller 180 outputs valve setpoints of valve W 526 to off, valve V 523 to off, and valve F 527 to on; thus establishing fluid communication between viable blood reservoir 221 and aspirate pump 175 through filter 535. System controller 180 outputs a negative aspiration setpoint to aspirate pump 175, thus causing reverse shaft rotation and flow in the reinfusion flow direction 298. Analytical instrumentation (photo-detector 280 and conductivity meter 380) provide system controller 180 with confirmatory data that liquid from viable blood reservoir 221 is statistically indistinguishable from viable blood. Viable blood is optionally reinfused during the course of a thrombectomy procedure to alleviate patient exsanguination.

A number of example thrombectomy operating modes have been disclosed; mode selection is based upon data from analytical instrumentation and/or differential viscometry of the aspirate. Inhomogeneous aspirate composition is inherent to thrombectomy procedures. At times during a thrombectomy procedure the catheter contents are substantially that of viable blood, at other times the catheter contents are an inhomogeneous mixture of thrombus and blood. As an example, the contents of a catheter may be 80% blood and 20% thrombus. The thrombus may be uniformly distributed along the length of the catheter, or the thrombus may be coalesced at particular locations along the length of the catheter. Herein, differential viscometry, includes measuring the “effective viscosity” of the contents of a catheter of inhomogeneous aspirate components which may or may not be spatially distinct from one another. Herein, viscometry correlates a measured pressure to the viscosity of a homogeneous fluid; the introduction of inhomogeneous fluids results in measuring the “effective viscosity” of the contents of the catheter.

The example thrombectomy operating modes disclosed herein are merely specific examples representative of the much broader realm of potential operating modes of any apparatus or system involving fluid transport.

FIG. 1C is a flowchart that details an example decision tree for selection of thrombectomy operating mode by system controller 180 based upon aspirate characteristic.

Initially within an example thrombectomy procedure, the catheter is filled with viable blood; the inflow sampling mode is executed. After sufficient sampling of viable blood, the viable blood aspirate characteristic is established by the calculation of SPC control chart limits for each analytical instrumentation system. The catheter is advanced to the next site; inflow sampling mode continues to monitor for a change in aspirate characteristic.

Inflow sampling mode is repeatedly executed until thrombus is detected by a change in aspirate characteristic that exceeds SPC control chart limits (Thrombus detected=yes), whereupon thrombus extraction mode is executed. Continuously or intermittently during thrombus extraction mode, data from analytical instrumentation are monitored for a change in aspirate characteristic. Thrombus extraction mode is continued until either (1) aspirate characteristic changes to viable blood (thrombus detected=no, catheter is advanced to the next site) or (2) thrombus extraction mode exceeds the prescribed cycle count (repeat n times).

If aspirate characteristic=thrombus for a cycle count exceeding n, a clog detection algorithm, e.g., pressure decay, is executed. If (clog detected=yes) the clog clearing mode is implemented. If (wall-adherent thrombus detected=yes) the radial direct impingement thrombectomy operating mode is executed. If (wall-adherent thrombus detected=no) the catheter is advanced to the next site.

The flowchart of FIG. 1B serves to illustrate, in rudimentary fashion, the data-driven execution of multiple thrombectomy operating modes within a single thrombectomy procedure. System controller 180 receives variable data from analytical instrumentation to determine the aspirate characteristic and subsequently executes a decision-tree flowchart such as depicted in FIG. 1B.

The thrombectomy system of FIG. 1A employs statistical inference to determine and respond to changes in the aspirate characteristic; the clinician is continuously apprised of aspirate characteristic through incremental audible or visual means. This greatly reduces the required clinician input, skill and attentiveness, thereby enhancing the clinician's ability to access multiple sites with improved endpoint determination within a single procedure.

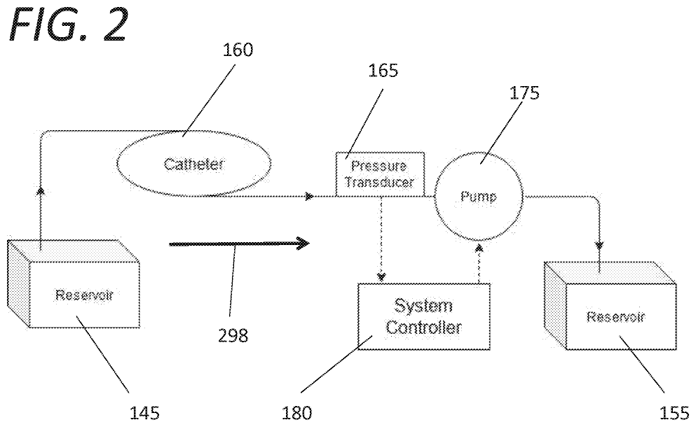

FIG. 2 shows the differential viscometer of the present invention in block diagram. Liquid is transferred from inlet reservoir 145 to discharge reservoir 155. Aspirate pump 175 draws liquid from inlet reservoir 145, through catheter 160, through pressure transducer 165 and ultimately to discharge reservoir 155. Inlet reservoir 145 and discharge reservoir 155 may be assumed to exist at atmospheric pressure without loss of generality; inlet reservoir 145 and discharge reservoir 155 may be common or otherwise in fluid communication. In other instances, discharge reservoir 155 may operate at pressure significantly different from inlet reservoir 145; this is exhibited by thrombectomy catheter systems employing an evacuated reservoir as the source of differential pressure causing aspirate flow. Without loss of generality, the inlet reservoir 145 is considered to be held constant at essentially atmospheric pressure; clinically however, inlet reservoir 145 may be at arterial or venous pressures during a thrombectomy procedure. Any associated, cyclic pressure fluctuations measured by pressure transducer 165 from physiological activity are acknowledged yet overlooked herein.

Fluid which is transferred through catheter 160 experiences a pressure drop between inlet reservoir 145 and pressure transducer 165. Pressure transducer 165 is therefore measuring a pressure that is less than atmospheric; this may be called vacuum or suction. Within a range of flow rates, the inlet port of aspirate pump 175 and pressure transducer 165 operate under vacuum; the magnitude of this vacuum is a function of liquid viscosity.

For any given liquid of viscosity u, aspirate pump 175 may be operated at multiple aspiration setpoints, each aspiration setpoint gives rise to a characteristic pump inlet pressure that is measured by pressure transducer 165. Increasing the aspiration setpoint of aspirate pump 175 will increase the flow rate of liquid through catheter 160; this increased flow rate generates an increase in viscous drag. Increasing the aspiration setpoint of aspirate pump 175 generates increased viscous drag in catheter 160 that increases the pressure drop between inlet reservoir 145 and pressure transducer 165.

It is instructive to compare the differential viscometer of FIG. 2 to an orifice plate flowmeter because both systems measure a flowing fluid property with at least one pressure measurement device, e.g., pressure transducer. The differential viscometer of FIG. 2 is comprised of a single detector (pressure transducer 165) and a controlled source of differential pressure (aspirate pump 175). Contrastingly, the orifice plate flowmeter requires two pressure transducers measure the differential pressure across a pressure drop (an orifice plate restriction) to subsequently calculate flow. An orifice plate flowmeter requires that the upstream and downstream pressures are simultaneously measured in order to subtract out any system pressure that may be present; thereby obtaining the differential pressure by subtraction.

A simplified working equation for an orifice plate flowmeter is presented in Eq. 1.

Q≈k\sqrt{√Δp} Eq. 1

Where: Q is the flow rate, k is a proportionality constant and Δp is the differential pressure across the orifice plate (or a length of a catheter). Flow is proportional to the square root of the differential pressure; pressure is measured at two locations to determine flow. Flow is an extensive property, being dependent upon mass (also time); pressure is an intensive property. Eq. 1 relates the intensive property pressure to the extensive property flow. Viscosity is an intensive transport property that is tacitly assumed constant in the range of the validity of Eq. 1.

In the differential viscometer of FIG. 2, inlet reservoir 145 is considered to be at constant pressure; therefore, ongoing pressure measurement at the upstream location (distal tip of catheter 160) is not required for differential viscometry. The unknown pressure of inlet reservoir 145 is initially determined by measuring the static pressure at pressure transducer 165 at zero flow; the pressure of inlet reservoir 145 is differentially compared to real-time, flowing pressure data from pressure transducer 165 to provide information about the viscosity of fluid contained within catheter 160. The static pressure of inlet reservoir 145 is measured at any time by the differential viscometer of FIG. 2 by stopping aspirate pump 175, and allowing the system to come to static equilibrium. Pressure transducer 165 measures the pressure of inlet reservoir 145 when the pump is stopped; the same pressure transducer 165 subsequently measures the differential pressure between inlet reservoir 145 and pressure transducer 165 through catheter 160. Subtracting the constant pressure of inlet reservoir 145 from subsequent pressure data from flowing liquid directly yields the flowing differential pressure in arbitrary units. In this case, the differential pressure is measured between a flowing liquid and the same stationary liquid. In other cases, the differential pressure is measured between different flowing liquids with a common static pressure of inlet reservoir 145.

The differential viscometer of FIG. 2 measures the viscosity of fluid in catheter 160 by measuring the differential pressure between inlet reservoir 145 and pressure transducer 165; this differential pressure is generated by aspirate pump 175. The differential viscometer of FIG. 2 sequentially calibrates inlet reservoir pressure (at zero flow) and determines the transport property viscosity (at varying rates of shear) by a methodology disclosed herein.

The differential viscometer shown in FIG. 2 employs aspirate pump 175 disposed between inlet reservoir 145 and discharge reservoir 155 which causes flow because a differential pressure exists along the length of catheter 160; inlet reservoir 145 and discharge reservoir 155 may operate at the same pressure, e.g., atmospheric, however a controlled source of differential pressure exists along the length of catheter 160. For large reservoirs at equal pressure, power input to the system through pump 175 is approximately equal to the viscous losses incurred in liquid transfer.

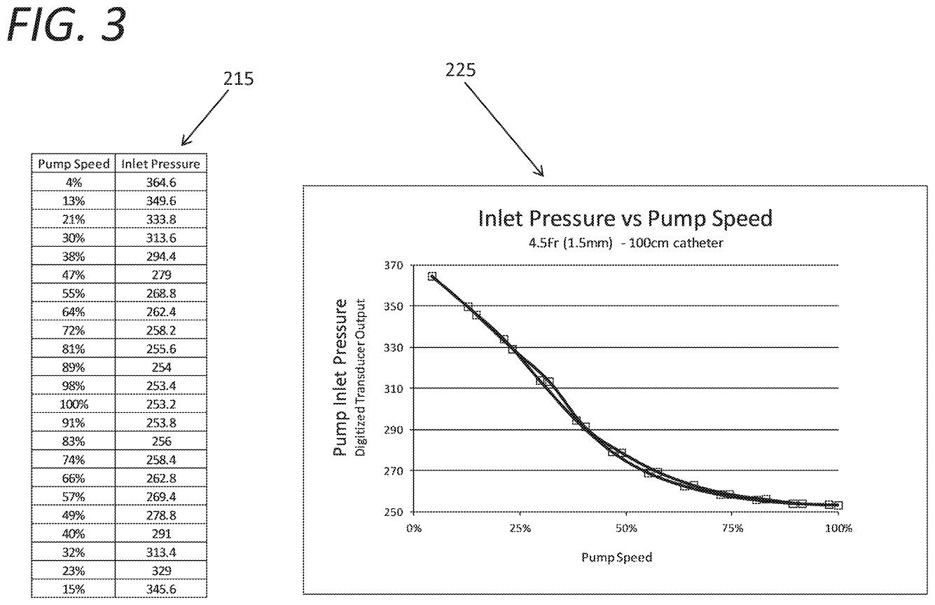

FIG. 3 shows tabular (inlet pressure data set 215) and graphical 225 representations of inlet pressure vs pump speed data collected for a representative 1.5 mm×100 cm catheter in water. Aspirate pump 175 was operated at increasing, then decreasing aspiration setpoints; the data are experimentally indistinguishable in either speed direction (there is a lack of hysteresis). These data are characteristic for a specific catheter 160; changing the length or diameter of catheter 160 will require new data collection and processing just as for any change in catheter dimension. Graph 225 may be referred to as a calibration curve; the catheter dimensions and liquid under test are known.

Pump inlet pressure vs speed data are collected in a straightforward and rapid manner; variable speed aspirate pump 175 is operated through a range of aspiration setpoints while corresponding inlet pressure data from pressure transducer 165 are collected and logged by system controller 180. As an example, aspirate pump 175 may be operated at aspiration setpoints of 0, 10, 20, 30, . . . 100% of full speed; at zero speed pressure transducer 165 is calibrated to a standard (substantially atmospheric or inlet reservoir) pressure. Data for inlet pressure data set 215 is collected in less than 30 seconds; inlet pressure data set 215 comprises the basis data set for the aspirate characteristic of water. SPC and control chart techniques are used to calculate the inlet pressure upper and lower control chart limits for an aspirate characteristic=water.

From graph 225 of FIG. 3, it is evident that increasing the pump speed (and flow rate through catheter 160) results in an increased differential pressure between inlet reservoir 145 and pressure transducer 165. The Hagen-Poiseuille equation (Eq. 2) is well suited to the relevant variables for viscometry: differential pressure, the length and diameter of catheter 160, and viscosity, e.g., 1 cP (1 mPa*s) for water.

$$Δ p = \frac{8 μ LQ}{π {R}^{4}} ; Q = \frac{π {R}^{4} Δ p}{8 μ L} ; μ = \frac{π {R}^{4} Δ p}{8 Q L} \qquad Eq . 2$$

Where Δp is the differential pressure (between inlet reservoir 145 and pressure transducer 165), Q is the volumetric flow rate, μ is the dynamic viscosity, L and R are the length and radius of catheter 160. The Hagen-Poiseuille equation (Eq. 2) relates intensive properties (μ and Δp) to the extensive property flow, Q. Setting the extensive property, Q, to be fixed at any value, (any aspiration setpoint) the relationship between the intensive properties is expressed in Eq. 3.

Δp≈Cμ Eq. 3

Where C is a proportionality constant (unrelated to the proportionality constant k of Eq. 1). On one hand, Eq. 1 states that flow is proportional to an algebraic function of the differential pressure; but this equation is valid with the premise that viscosity remains constant. On the other hand, Eq. 3 states that the liquid viscosity is linearly proportional to the differential pressure; but this equation is valid with the premise that flow remains constant. During the course of a thrombectomy procedure, the anticipated aspirate composition is a heterogeneous and time-dependent mixture of liquid, semi-solid (gelatinous) and solid components. The premise that aspirate viscosity remains constant throughout a thrombectomy procedure must be rejected.

Comparing Eq. 1 and Eq. 3, the differential pressure (Δp) is proportional to both the extensive property flow (Q) and the intensive transport property viscosity (μ), although under very different conditions. Eq. 1 requires that the viscosity is held constant, whereas Eq. 3 requires that flow is held constant. In the differential viscometer of FIG. 1A, flow is continuously variable and is an unknown function of aspiration setpoint and liquid viscosity.

The objective of a thrombectomy procedure is to aspirate a maximum quantity of thrombus mixed with a minimum quantity of viable blood; the proportion of each component is visible to the clinician by observing the contents of the waste reservoir 226. A favorable ratio of thrombus to viable blood requires that thrombotic components are preferentially aspirated; these thrombotic components are necessarily of viscosity that is greater than blood. A successful thrombectomy procedure is anticipated to aspirate liquid of viscosity significantly greater than that of blood. In a clinical thrombectomy setting, rapidly changing aspirate viscosity degrades the accuracy of an orifice plate flowmeter while the differential viscometer of FIG. 2 accurately measures the differential viscosity of the viscous aspirate.

An orifice plate style differential pressure flowmeter is comprised of two pressure transducers and is ill-suited to the flow-quantization analysis of liquids of varying viscosity. Contrastingly, the differential viscometer of FIG. 2 is comprised of only a single pressure transducer along with a controlled source of differential pressure to elicit clinically-relevant determination of the viscous aspirate characteristic.

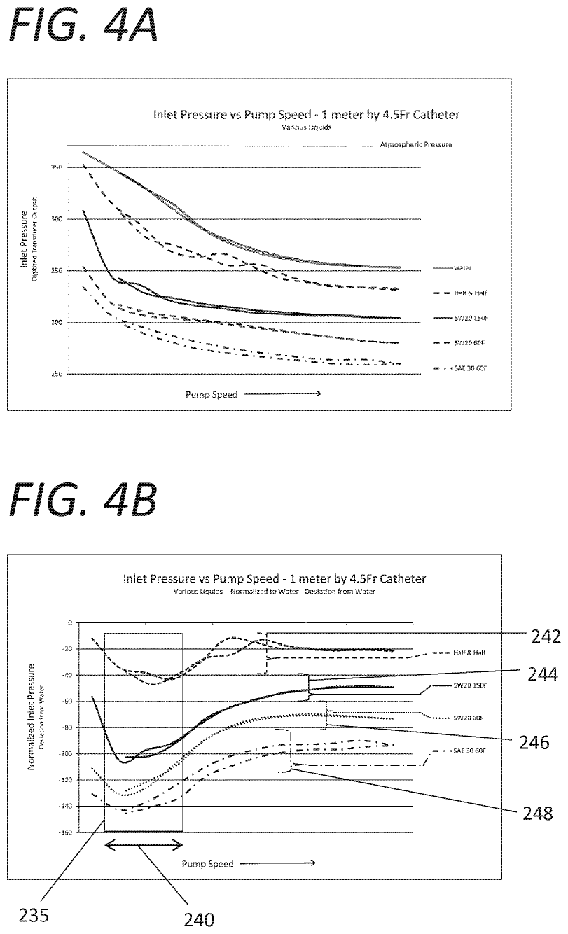

FIG. 4A is a graphical representation of readily-available liquids analyzed in the differential viscometer of FIG. 2 equipped with a representative catheter 160 which is 1.5 mm diameter and 1 m in length. The liquids are water, half and half, 5W20 motor oil (at 60 °F and 150 °F) and SAE 30 motor oil (at 60 °F). Each of the liquids generates a distinct pressure-speed curve that is readily distinguished from the other curves. The differential viscometer of FIG. 2 thus measures viscosity not in engineering units (cP, mPa*s, cS, etc.), but in arbitrary pressure units (at a given aspiration setpoint). In engineering units, water has viscosity of approximately 1 cP and SAE 30 motor oil has viscosity of approximately 240 cP (at 60 °F); the graph of FIG. 4A shows minimum arbitrary pressure units of approximately 255 for water and 160 for SAE 30 motor oil (at maximum aspiration setpoint). The pressure units are arbitrary, discretized pressure transducer output with approximately 375 being atmospheric pressure. A pressure transducer or an analog to digital converter with different specifications results in different pressure measurements, just as changing the dimensions of catheter 160 will.

FIG. 4B differs from FIG. 4A in that the data are “normalized” to water by subtracting the inlet pressure of water from the inlet pressure measurement for each liquid. The curves may be normalized to any chosen liquid, e.g., blood. The pump inlet pressure curves of FIG. 4B show a maximum deviation from the normalization liquid (water) in the rectangular maximum sensitivity region 235, corresponding to a maximum sensitivity domain 240. The differential viscometer of FIG. 2 exhibits varying sensitivity at varying pump speed; operating pump 175 outside of the range of maximum sensitivity domain 240 results in diminished sensitivity of the differential viscometer of FIG. 2. Meaningful data may be collected at any pump speed, however operation of aspirate pump 175 within the maximum sensitivity domain 240 results in greatest sensitivity of the differential viscometer of FIG. 2. Aspirate sampling with maximum instrument sensitivity is accomplished at a low flow rate; this is advantageous during a thrombectomy procedure wherein the objective is to minimize the loss of viable blood.

The normalization of data with respect to water is an example of a differential viscometry calculation by subtraction, which retains arbitrary units. In FIG. 3, inlet pressure data table 215 expresses the pressure in arbitrary units (atmospheric pressure ≈375 arbitrary pressure units). Furthermore, pump speed is expressed as % full scale; also an arbitrary unit. The descriptor of any data point might therefore be 268.8 pressure units at 55% pump speed. Subsequently measuring a different fluid measuring 200 pressure units (at the same 55% pump speed), a subtractive differential viscometry calculation yields a difference of 68.8 pressure units. The arbitrary unit is retained in subtraction. Another example is differential viscometry calculation by division, which yields relative units. For example, the data from a first liquid of viscosity 300 pressure units and a second liquid of 150 pressure units are divided; this yields a viscosity ratio of 2:1 or the viscosity of the second liquid is 200% the viscosity of the first liquid. This ratio is dimensionless and expressed in relative units. Conversion of arbitrary or relative units to engineering units, e.g., cP, SUS, cSt, etc., may be performed as required, however this is not necessary for reducing the present invention to practice.

The range of data in graph of FIG. 4B has been subdivided into sub-ranges delimiting the heterogeneous data acquired from each of the liquids subjected to differential viscometry. Sub-range HH 242 shows an example data range corresponding to Half & Half at a first pump speed setpoint. Subsequent experimental data generally populating the limits of sub-range HH 242 may be assigned (fluid characteristic=half and half). Sub-range 5W20@150 244 shows an example sub-range of data corresponding to 5W20 at 150 °F at a second pump speed setpoint. Sub-range 5W20@60 246 shows an example sub-range of data corresponding to 5W20 at 150 °F at a third pump speed setpoint. Sub-range SAE30@60 244 shows an example sub-range of data corresponding to SAE 30 motor oil at 60 °F at a fourth pump speed setpoint. For clarity in FIG. 4B, each liquid shows only one sub-range at a selected pump speed setpoint; the selected pump speed setpoints differ for clarity in identifying each sub-range. In practice, the data range is subdivided into sub-ranges at each pump speed setpoint; a plurality of sub-ranges, corresponding to a plurality of pump speed setpoints are thereby determined. Each pump speed setpoint is a slice of the domain (with a characteristic range), the range at each pump speed setpoint is subdivided into sub-ranges. The limits of each such example sub-range may be determined by statistical means, a fixed interval or any mathematical function.

FIG. 4C illustrates sub-ranging of the data by fluid characteristic at 5 pump speeds. At a pump speed of 20%, the range of data for water is 392.5 to 400 arbitrary pressure units. These data are shown in the sub-range Water @ 20% 110. The fluid characteristic for subsequent data lying in this sub-range is water (fluid characteristic=water). Data of sub-range H&H @ 40% 115 are shown to range from 350 to 360 arbitrary pressure units. The fluid characteristic for subsequent data lying in this sub-range is half & half (fluid characteristic=half & half). Data of sub-range Warm 5W20 @60% 120 are shown to range from 292.5 to 307.5 arbitrary pressure units. The fluid characteristic for subsequent data lying in this sub-range is Warm 5W20 (fluid characteristic=Warm 5W20). Data of sub-range Cold 5W20 @ 80% 125 are shown to range from 220 to 240 arbitrary pressure units. The fluid characteristic for subsequent data lying in this sub-range is Cold 5W20 (fluid characteristic=Cold 5W20). Data of sub-range SAE 30 @ 100% 130 are shown to range from 95 to 125 arbitrary pressure units. The fluid characteristic for subsequent data lying in this sub-range is SAE 30 (fluid characteristic=SAE 30).

The act of sub-dividing the range into sub-ranges transforms variable data (pump inlet pressure) into attribute data (half & half, SAE 30 motor oil, blood, thrombus, etc.). The variable data are retained for any purpose, however the variable data are used to establish attribute data sub-ranges. Subsequent experimental data may thereby be characterized by comparing experimental data to the pre-established attribute data sub-ranges, each sub-range is assigned an fluid characteristic. As examples, unknown fluids are measured for viscosity in the viscometer of FIG. 2. At pump speed 40%, if the arbitrary pressure unit data is 310, then the unknown fluid is assigned (fluid characteristic=SAE 30). At pump speed 80%, if the arbitrary pressure unit data is 320, then the unknown fluid is assigned (fluid characteristic=water). Thereby an unknown liquid may be characterized based upon the attribute data sub-range that the unknown liquid data generally populates; the fluid characteristic or aspirate characteristic is thereby determined.

The foregoing data are signal conditioned by averaging a number of data points collected at each pump speed setpoint. After each change of pump setpoint, the system is allowed to equilibrate for a short period of time (0.1 second to 2 seconds) prior to data collection. At each pump speed setpoint, for each liquid, a statistically significant number of data points may be readily collected for the determination of mean, standard deviation, range, etc. Each data point of the preceding graphs and tables is the data mean over the measurement interval; standard deviation and range are also calculated and stored. Thus a large quantity of variable data is available for the application of statistical techniques employed in sub-dividing the data range into sub-ranges for attribute classification.

FIG. 4B and FIG. 4C illustrate the ability of the differential viscometer of FIG. 2 to differentially measure the viscosity across a range of shear rates. Increasing the pump speed differentially measures the viscosity of the liquids under test at correspondingly increasing shear rates. This imparts the ability of the differential viscometer of FIG. 2 to measure not only viscosity but rheology (viscosity at varying rates of shear). Also noteworthy is evidence of shear hysteresis; SAE 30 motor oil and half and half exhibit shear hysteresis whereas 5W20 motor oil (like water) does not exhibit appreciable hysteresis. Shear hysteresis is evident in liquids where the pump inlet pressures are different as the aspiration setpoints are raised and then lowered; the line trace does not overlay itself on the return path. Liquid properties such as shear hysteresis and other rheological data are inherently and readily available from routine operation of the differential viscometer of FIG. 2.

Rheological data are valuable in the quality assessment of liquids such as motor oil where viscosity breakdown (at different shear rates) provides greater information regarding the oil's ability to lubricate under high pressure and/or rates of shear. FIG. 4B further illustrates that liquids may be tested at different temperatures; for instance, an oil sample may be subjected to temperature variations (such as in a gearbox or crankcase). In this case thermal viscous breakdown may be detected and quantified simply by comparing the liquid viscosity (at a particular temperature and shear rate) to a reference liquid in real time. An obvious implication of this is that oil quality may be continuously monitored over time to detect breakdown of the oil's lubricating qualities; oil breakdown may be detected in real time to alert the equipment operator to perform timely oil changes. Timely oil changes ensure maximum equipment service life with minimum maintenance costs and downtime.

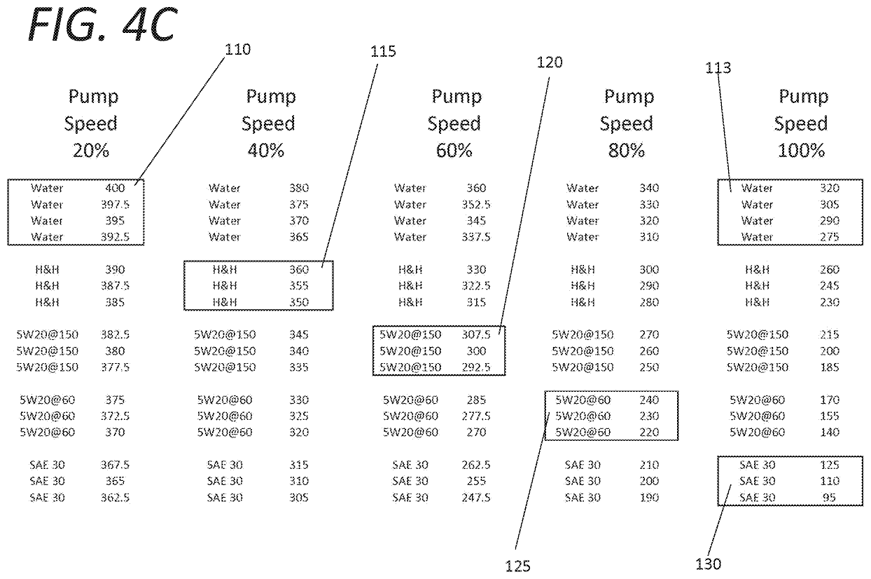

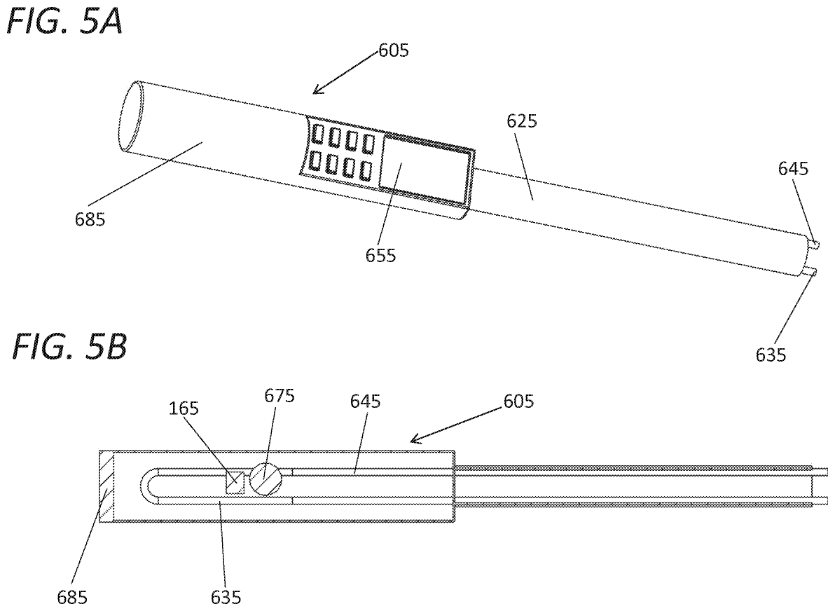

FIG. 5A depicts an oblique view of a practical embodiment of the differential viscometer of FIG. 2 comprising probe viscometer 605 for permanently-installed or field use in a variety of scientific, industrial, maintenance and original equipment applications. Probe handle 685 illustrates that the device may be handheld; however probe viscometer 605 may be mounted in a reservoir or process stream, such as by a flange or pipe tap connection. Keypad display 655 allows for user input and read-out of data, system controller 180 is integral to keypad display 655. Liquid enters probe viscometer 605 through the length of probe inlet tube 635 and is subjected to differential viscometric analysis. Subsequent to differential viscometric analysis, liquids exits probe viscometer 605 through probe outlet tube 645. Probe inlet tube 635 and probe outlet tube 645 are covered by probe cover 625. Probe inlet tube 635 comprises a number of interchangeable components of different length and/or diameter such that the measurement viscosity range is extended to include viscous as well as inviscid liquids.

FIG. 5B shows probe viscometer 605 in cutaway view revealing the internal components and fluid pathway. Probe inlet tube 635 exhibits a longer and more convoluted pathway compared to probe outlet tube 645 such that the pressure drop across probe inlet tube 635 is sufficient for accurate pressure and viscometric data to be collected. Operation of probe pump 675 within maximum sensitivity domain 240 is preferred for instrument accuracy; this is facilitated by the proper selection of the diameter and length of probe inlet tube 635 corresponding to the viscosity range of liquid to be analyzed. Probe pump 675 is the controlled source of differential pressure; pressure transducer 165 is located in inlet tube 635 such that probe pump 675 inlet pressure is measured. Pressure transducer 165 is shown immediately adjacent to probe pump 675, which is a preferred configuration; locating pressure transducer 165 at any other location along probe inlet tube 635 provides similar, though less accurate results.

Probe viscometer 605 of FIG. 5A and FIG. 5B is comprised of at least one methodology of analytical instrumentation including viscometry, conductivity, pH, temperature, absorbance, etc., thereby a plurality of data may be concurrently transmitted or displayed, e.g., viscosity and temperature. Probe viscometer 605 is comprised of at least one methodology of data input and output, including keypad, visual display, Bluetooth, USB data port, etc.

FIG. 6 shows a section view of conductivity analyzer 380 as an example of analytical instrumentation for determining an aspirate characteristic of aspirate under test 285. Flow is from left-to-right 298, with upstream aspirate 291 flowing toward conductivity analyzer 380; downstream aspirate 296 has been analyzed for conductivity and has been characterized, e.g., as viable or thrombus-laden blood. Upstream electrode 315 and downstream electrode 325 are at different voltage potential (1 mV to 5V) causing a small current (1 μA to 100 mA) to flow through aspirate under test 285; the voltage potential may be comprised of direct or alternating current, and the voltage potential may be otherwise time-dependent. The electrically conductive pathway between the conductivity analyzer 380 and the patient bloodstream is easily minimized or eliminated by locating the conductivity analyzer 380 downstream of aspirate pump 175.

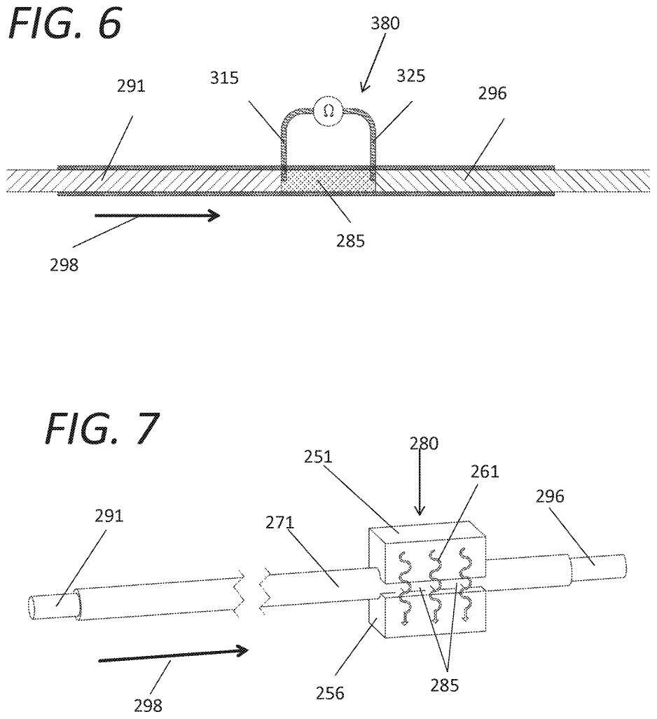

FIG. 7 shows an oblique view of photo-detector 280 encapsulating clear tubing 271 such that electromagnetic radiation, e.g., light 261, passes through aspirate under test 285 such that the local aspirate is subject to absorption photometry. Flow is shown left-to-right 298, with upstream aspirate 291 flowing toward photo-detector 280; downstream aspirate 296 has been analyzed for absorbance (% transmittance) and has an aspirate characteristic determined, e.g., blood or thrombus. Emitter unit 251 comprises a source (or array of sources) of light 261 with directional arrows indicating that light 261 flows through clear tubing 271 and aspirate under test 285. Detector unit 256 comprises an electromagnetic radiation detector (or array of detectors) that detect light 261 intensity subsequent to partial absorption by clear tubing 271 and aspirate under test 285. Photo-detector 280 is representative of a broad array of analytical instrumentation systems for the discriminative determination of the aspirate characteristic of aspirate under test 285; these data are collected and analyzed by system controller 180 for subsequent system response.

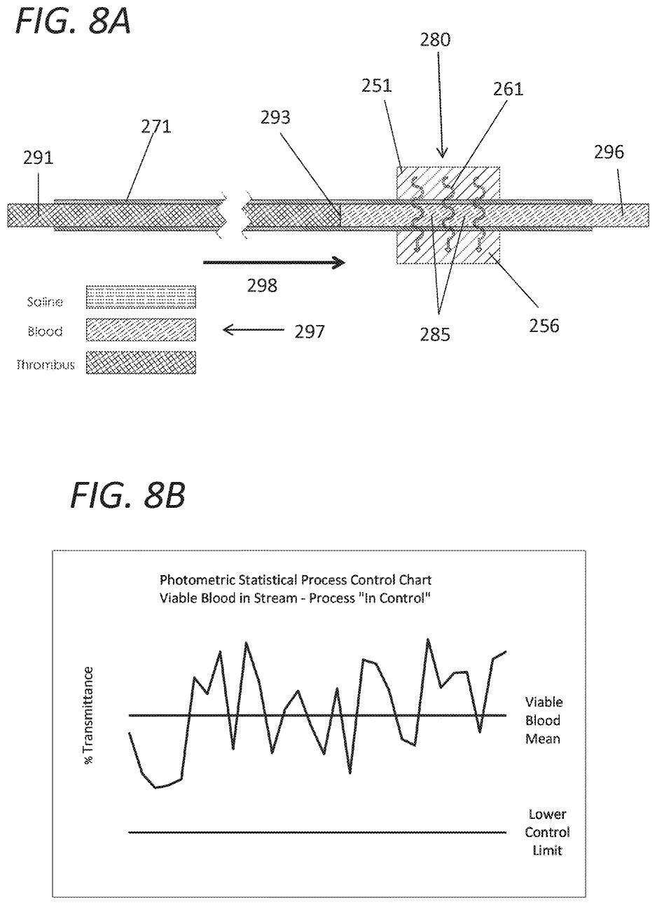

FIG. 8A shows a section view of photo-detector 280; upstream aspirate 291 (identified as thrombus from hatch legend 297) flows toward photo-detector 280, flow is left-to-right 298. Thrombus in upstream aspirate 291 is shown isolated from blood in downstream aspirate 296 by interface 293. Downstream aspirate 296 is of the same composition (blood) as aspirate under test 285, photo-detector 280 is measuring the absorption (% transmission) of blood in FIG. 6. Light 261 from emitter unit 251 passes through clear tubing 271 (twice) and aspirate under test 285 before impinging on detector unit 256. Flow is considered to be continuous (under most thrombectomy operating modes), therefore each data sample collected by photo-detector 280 represents a spatial slice of aspirate; and the aspirate composition is analyzed in a manner similar to the measurement of parts coming off of a production line. SPC and control chart techniques are routinely used in a production line to identify non-conforming product; similar control chart techniques are employed in the present invention to identify and segregate blood and thrombus as the aspirate characteristic changes in time.

FIG. 8B graphically depicts the application of SPC and control chart techniques to track the data history of aspirate under test 285. At some time during a thrombectomy procedure, catheter 160 tip traverses non-diseased portions of the vasculature where only viable blood is aspirated; this period of time is used to establish the mean, standard deviation and range of data from photo-detector 280. Control limits calculated from these data to establish a lower % transmittance (or upper absorption) threshold that is identified as the LCL of % transmittance for the patient blood. % Transmittance data between the UCL and LCL are statistically indistinguishable from that of viable blood in the process stream; statistically, the data of the process stream is termed “in control.” FIG. 8B shows an “in control” process; the requirement for assigning (aspirate characteristic=viable blood) is met in FIG. 8B.

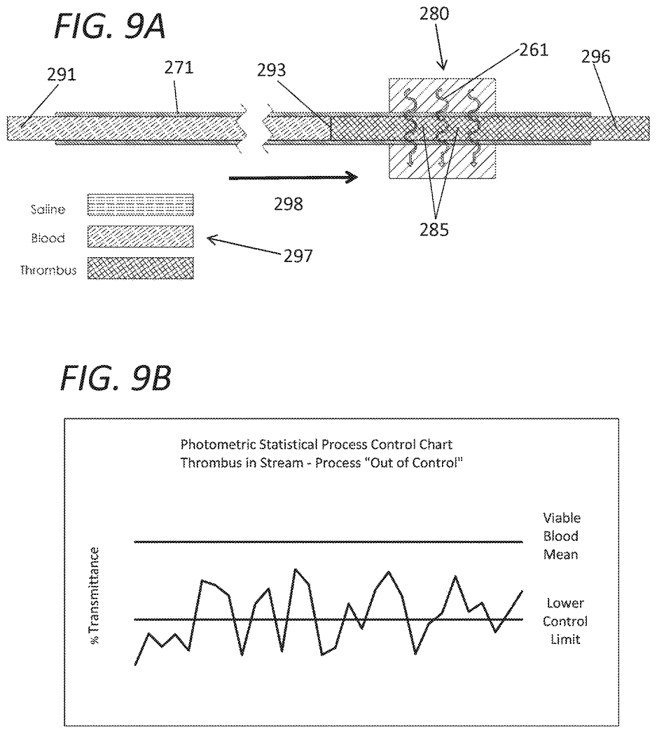

FIG. 9A is analogous to FIG. 8A except that the positions of blood and thrombus have been reversed. In FIG. 9A the composition of aspirate under test 285 is thrombus; photo-detector 280 is sampling a process stream with a % transmittance less than that of blood (absorption is greater than that of blood).

FIG. 9B is analogous to FIG. 8B except that the process is statistically “out of control,” because the process stream data populate outside the LCL. The threshold is met to change the aspirate characteristic (aspirate characteristic≠viable blood). In an analogous manufacturing process, data collected from measured parts indicate that the process is “out of control” and that adjustments to the manufacturing equipment are required. In the thrombectomy process stream, the aspirate characteristic has changed and system controller 180 subsequently updates the thrombectomy operating mode.

Data from the differential viscometer of FIG. 2 and conductivity analyzer 380 of FIG. 6 may be treated in a manner similar to that of photo-detector 280 of FIG. 7 and FIG. 8A. Conductivity analyzer 380, photo-detector 280 and the differential viscometer of FIG. 2 represent three examples of analytical instrumentation systems located in the process stream of the thrombectomy system of the present invention. Other methodologies to analytically detect the aspirate characteristic of the thrombectomy process stream are anticipated that are equally-well captured by the intended scope of the present invention.

The foregoing discloses two example techniques for determining the aspirate characteristic: SPC control charting and sub-ranging; both techniques are based upon analysis of previously-collected data for one or more different liquids. SPC control charting is presented as a technique to detect process drift in terms of variable-data (e.g., aspirate characteristic transitions to becoming statistically distinguishable from blood). Sub-ranging is presented as a technique to otherwise quantify the drift, in attribute terms, including across a plurality of aspirate characteristics, e.g., (aspirate characteristic=blood) transitions to (aspirate characteristic=clog). Both attribute and variable data are employed herein.

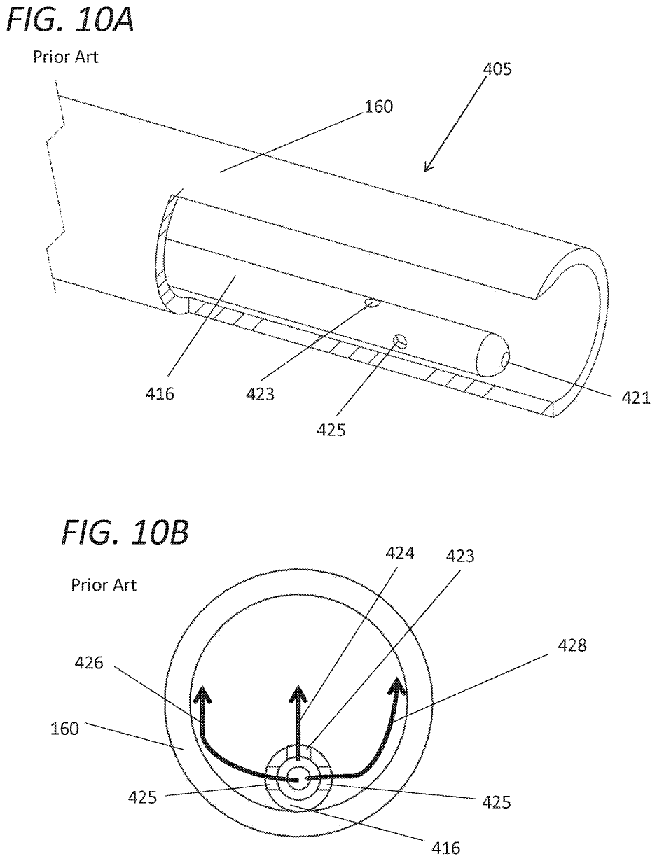

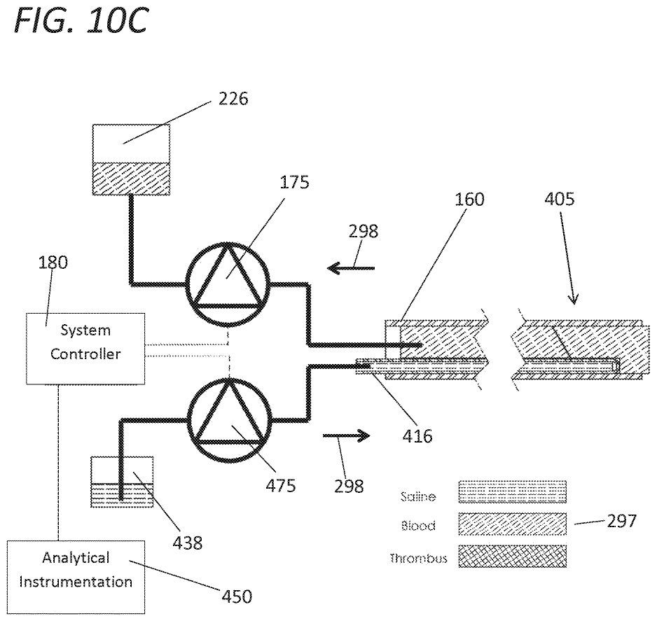

FIG. 10A shows an oblique, partial cutaway view of an embodiment of clog clearing tip 405 incorporating a hydrodynamic tubing 416 that provides a fluid pathway for the infusion of liquid to modify the composition and flow characteristics throughout the length of catheter 160. Catheter 160 comprising clog clearing tip 405 is shown as a variant of any standard or custom dual-lumen tubing. Hydrodynamic tubing 416 of catheter 160 with clog clearing tip 405 is depicted with three distinct nozzles from which flow emanates: axial nozzle 421, tangential nozzle 425, and radial nozzle 423; one of each variant is depicted in FIG. 10A, in practice, any number of nozzles may be present.

FIG. 10B shows an end-view of catheter 160 with clog clearing tip 405, showing representative fluid pathways outward from hydrodynamic tubing 416. Radial jet 424 emanates from radial nozzle 423. Tangential jet CW 426 and tangential jet CCW 428 emanate from tangential nozzles 425 disposed about the hydrodynamic tubing 416. Tangential jets 426, 428 induce rotational flow about the catheter 160 axis. Rotational flow enhances mixing of aspirate with hydrodynamic liquid, which is typically saline-based; this mixing of aspirate and saline increases local flow velocity while reducing the viscosity of the aspirate. Reducing the aspirate viscosity increases the flow rate of the diluted aspirate. Radial jet 424, emanating from radial nozzle 423, impinges upon catheter 160 at an angle that is approximately perpendicular. Hereafter, only radial nozzle 423 and radial jet 424 are considered.

FIG. 10A and FIG. 10B show a representative embodiment of a clog clearing tip 405 that does not appreciably deviate from embodiments of prior art. The structure depicted serves only as a contributory component of the present invention: automated orchestration of multiple independent subsystems (analytical instrumentation, pumps and valves) to result in maximum clinical efficacy while simultaneously requiring minimum operator expertise and attentiveness.

FIG. 10C shows the fluid communication pathways of clog clearing tip 405, integrated into the thrombectomy system of the present invention, comprised of a system controller 180, analytical instrumentation 450 and a plurality of reservoirs and pumps under system control. Aspirate flow direction 298 and hatch legend 297 indicate the relevant flowfield information. Infusion pump 475 is in fluid communication with saline reservoir 438 and the lumen of hydrodynamic tubing 416. Aspirate pump 175 is in fluid communication with the lumen of catheter 160 and waste reservoir 226. Both aspirate pump 175 and infusion pump 475 are independently controlled by system controller 180. System controller 180 collects and analyzes data from any number of analytical instrumentation 450 devices or systems to infer the aspirate characteristic, and subsequently provide effective therapeutic treatment dependent upon analyses conducted upon the contents of catheter 160. While prior art may incorporate multiple pumps or vacuum sources operating simultaneously, prior art does not anticipate the incorporation of analytical instrumentation 450 that provides continuously updated information regarding the aspirate characteristic of the contents of catheter 160. While prior art may incorporate some of the parts and components illustrated in FIG. 10C, the inventive step is to provide automated control of the inflow and outflow to catheter 160 based upon continuously-updated data, including aspirate characteristic, from analytical instrumentation 450.

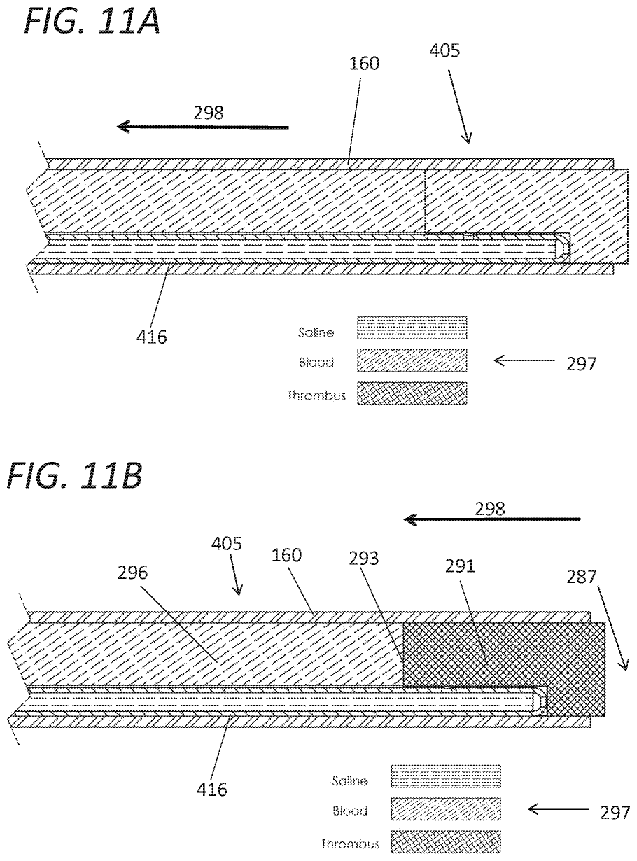

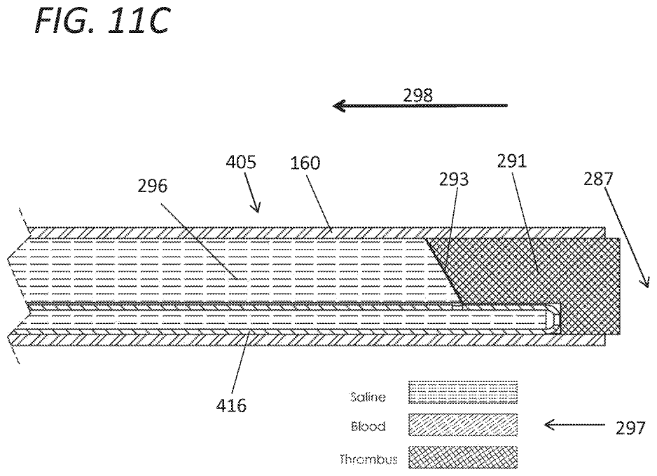

FIG. 11A shows a section view of an embodiment of clog clearing tip 405 at the distal end of catheter 160 which illustrates a relevant clinical situation wherein only blood comprises the depicted aspirate. Data from analytical instrumentation 450 transmitted to system controller 180 are used to assign (aspirate characteristic=blood).

FIG. 11B shows a section view of clog clearing tip 405 at the distal end of catheter 160 which illustrates a relevant clinical situation wherein blood comprises downstream aspirate 296 and thrombus comprises upstream aspirate 291, the blood and thrombus are separated by interface 293. Thrombus in upstream aspirate 291 is impeding flow through catheter 160. The inlet pressure of aspirate pump 175 will decrease and system controller 180 will determine (aspirate characteristic #blood).

At least 2 cases may arise: viscous, flowing aspirate is slowly traversing catheter 160, or a clog may reduce the flow to negligible. The two cases may be differentiated by system controller 180 by means of pressure decay analysis. Aspirate pump 175 setpoint is updated by system controller 180 from a high setpoint (generally greater than 70%) to a low setpoint (generally less than 30%) in a period of time generally less than 1 second. The length of time required for the inlet pressure of aspirate pump 175 to equilibrate to the pressure of the inlet reservoir 145 is deterministic of the presence and magnitude of flow through catheter 160. Pressure decay analyses are implemented to differentiate viscous aspirate from a clog, based upon the time required for the stopped aspirate pump 175 inlet pressure to equilibrate to the inlet reservoir 145 pressure. If the pressure decay time exceeds a threshold value, aspirate characteristic=clog is assigned by system controller 180; otherwise, aspirate characteristic=thrombus is assigned. FIG. 11B is considered herein to be of aspirate characteristic=clog.

An example control strategy to clear a clog (aspirate characteristic=clog) is to execute a saline infusion mode whereby saline is infused into the distal end of catheter 160 through hydrodynamic tubing 416 by executing any number neutral aspiration cycles. FIG. 11B shows blood as downstream aspirate 296; blood is more viscous than saline. Executing a saline infusion mode will replace saline for blood in downstream aspirate 296; this provides an inviscid length of liquid within catheter 160; this inviscid length may comprise a significant portion of the total length of catheter 160. A viscous downstream aspirate 296 (blood, thrombus) inhibits flow at any rate due to frictional losses; an inviscid downstream aspirate 296 (saline) flows more freely along the length of catheter 160 such that flow is achieved at lower differential pressure. This permits a greater differential pressure to be developed between interface 293 and intravascular freestream 287.

FIG. 11C shows a section view of clog clearing tip 405 at the distal end of catheter 160 that illustrates a relevant clinical situation following execution of saline infusion mode. Interface 293 is shown having moved distally which reduces the volume of thrombus in upstream aspirate 291. Furthermore, the composition of downstream aspirate 296 is shown to be substantially saline, which provides a greater differential pressure between interface 293 and the intravascular freestream 287 than would blood as in FIG. 11B.

The saline infusion mode may occur at low infusion pressure (3 psi to 25 psi) or at high infusion pressure (25 psi to 10,000 psi); at low infusion pressure, radial jet 424 does not possess sufficient momentum to erode or macerate any solid thrombus in the pathway of the radial jet 424. At higher infusion pressures, the liquid momentum of radial jet 424 becomes sufficient to erode or macerate any thrombotic material directly in the path of the jet. Comparing FIG. 11B and FIG. 11C, note that interface 293 has rotated and translated; some thrombus has been eroded away during the course of the saline exchange. This process may be accomplished at low infusion pressure if the thrombotic material is soft and compliant, while greater infusion pressure is required to treat more hard, dense or fibrous thrombus.

Infusion pump 475 may be operated at low infusion setpoint to slowly infuse saline into the aspirate or at high infusion setpoint to macerate thrombus. A piston pump transfers a characteristic volume with each pump cycle. A piston pump driven by a stepper motor is capable of delivering a specific volume of liquid in a specific period of time, system controller 180 thereby exhibits setpoint control over infusion pressure. Each cycle of a piston pump is independent of prior and successive cycles executed by system controller 180; a single, high infusion setpoint pump cycle may be interspersed an otherwise continuous succession of lower infusion setpoint cycles. High-velocity jets of short-duration infusion cycles (1 to 100 cycles) interspersed with other infusion flow rate regimes is another example of a thrombectomy operating mode available to system controller 180.

Aspirate pump 175 and infusion pump 475 respond independently to their respective setpoints to provide any combination of aspirate pump 175 inlet pressure and infusion pump 475 discharge pressure at radial nozzle 423. Operating infusion pump 475 at increasing setpoint increases the flow and pressure through hydrodynamic tubing 416; this leads to a correspondingly greater velocity of radial jet 424. High-velocity radial jet 424 is capable of significant tissue damage if it is not contained by catheter 160; system controller 180 controls the speed and count of each cycle of infusion pump 475.

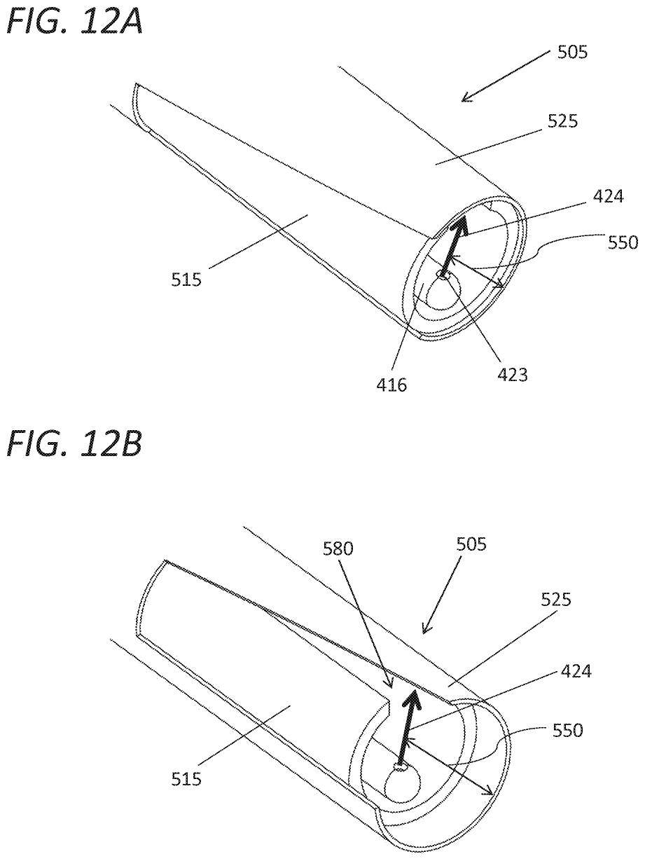

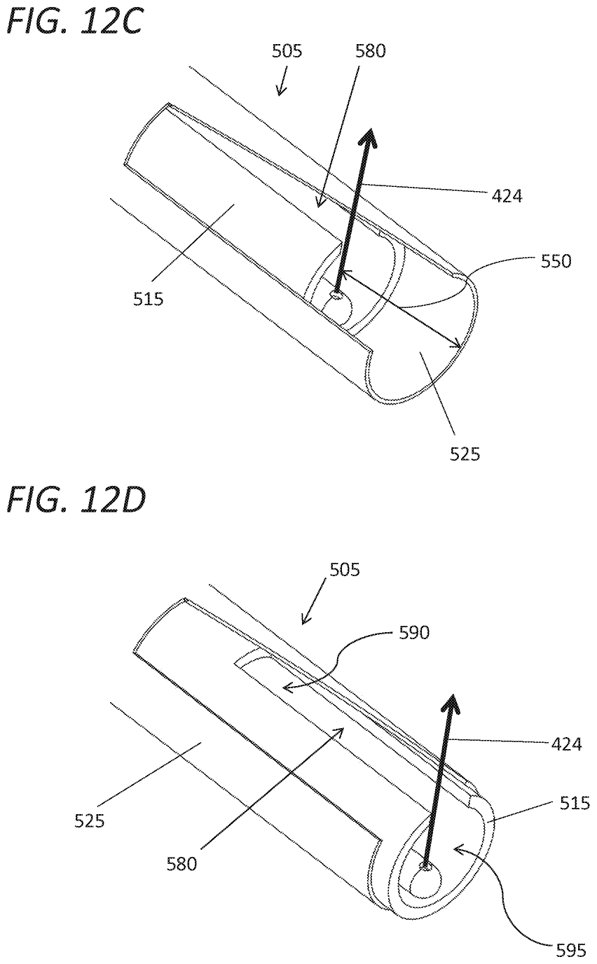

FIG. 12A shows an oblique view of an embodiment of variable aperture tip 505 shown in a first configuration, exhibiting a right-circular cylindrical shell at the distal terminus. Functionally, the configuration shown in FIG. 10A is virtually indistinguishable from Clog Clearing Tip 405 (of FIG. 11A, FIG. 11B, and FIG. 11C) comprising only a single radial nozzle 423 disposed distally upon hydrodynamic tubing 416. Variable aperture catheter 515 and variable aperture sheath 525 form a complete circumferential boundary to allow for axial inflow and outflow. Hydrodynamic tubing 416 terminates in close proximity to the distal terminus of variable aperture tip 505; radial jet 424 impacts variable aperture sheath 525. Radial jet 424 is located a characteristic axial distance away from the distal terminus of the variable aperture tip 505; this distance is shown as jet tip distance 550.

FIG. 12B shows variable aperture tip 505 of in a second configuration achieved by changing the rotational and axial positions of variable aperture sheath 525 with respect to variable aperture catheter 515. Rotation of variable aperture sheath 525 opens or closes aperture 580 while axial motion of the same component affects both the size of aperture 580 and the jet tip distance 550. As shown in FIG. 12B, radial jet 424 impacts variable aperture sheath 525 in the vicinity of aperture 580; a portion of radial jet 424 may escape through aperture 580 to directly impinge upon surrounding intravascular tissue.

FIG. 12C shows variable aperture tip 505 in a third configuration that exhibits a long jet tip distance 550 and aperture 580 sufficiently open for radial jet 424 to impinge directly upon surrounding tissue within the intravascular freestream.

FIG. 12D shows variable aperture tip 505 in a fourth configuration exhibiting fully open aperture 580; variable aperture sheath 525 is withdrawn axially with respect to variable aperture catheter 515. Inflow and outflow characteristics of variable aperture catheter 515 are selectable by means of any combination of axial and rotational motions of variable aperture sheath 525 with respect to variable aperture catheter 515.

FIG. 12C and FIG. 12D illustrate configurations suitable for radial direct impingement mode; aperture 580 is open to permit radial jet 424 to directly impinge upon surrounding tissue in the intravascular freestream. An example of the radial direct impingement mode consists of: variable aperture actuator setpoint is updated to such that aperture 580 is in the open configuration; infusion setpoint is updated to a level appropriate for maceration of anticipated thrombus characteristic, infusion pump 475 executes a number of outflow cycles to dislodge or macerate thrombus. Subsequently, aspiration setpoint is increased to evacuate the dislodged thrombus.

In the radial direct impingement mode, a limited number of cycles of high-velocity radial jet 424 are executed for maceration of wall-adherent thrombus; this maceration will generate a limited volume of thrombotic debris to be aspirated during the forthcoming aspiration cycles. The combination of intermittent cycles of high infusion setpoint, high-velocity radial jet 424 with concomitant, high aspiration setpoint cycles and radial inflow are used to effectively macerate and subsequently aspirate wall-adherent thrombus in a systematic manner. The quantity of thrombotic debris released with each infusion pump cycle is small; interspersing a number of aspirate pump cycles with a number of infusion pump cycles permits efficient thrombus evacuation. Thrombotic debris are thereby efficiently aspirated by intermittently dislodging and subsequently aspirating only a small quantity of thrombotic debris within a few cycles of the radial direct impingement mode.

Tissue (blood, thrombus) inflow to variable aperture tip 505 (in some configurations) is comprised of both axial inflow 595 and radial inflow 590. Catheters typically exhibit only a single inflow pathway, and the inflow is usually axial; catheters with radial inflow/outflow also exist, though less frequently. Variable aperture tip 505 provides selectable, alternate inflow/outflow pathways that range from substantially axial inflow 595 to a combination of radial inflow 590 and axial inflow 595. Radial inflow 590 is advantageous in the treatment of wall-adherent thrombus. As aperture 580 is opened to permit radial inflow 590 there is a significant decrease in axial inflow 595 because aperture 580 is downstream of the distal tip. Radial inflow 590 is preferentially exhibited over axial inflow 595 because there is only a very small differential pressure between the axial catheter aperture and intravascular freestream.

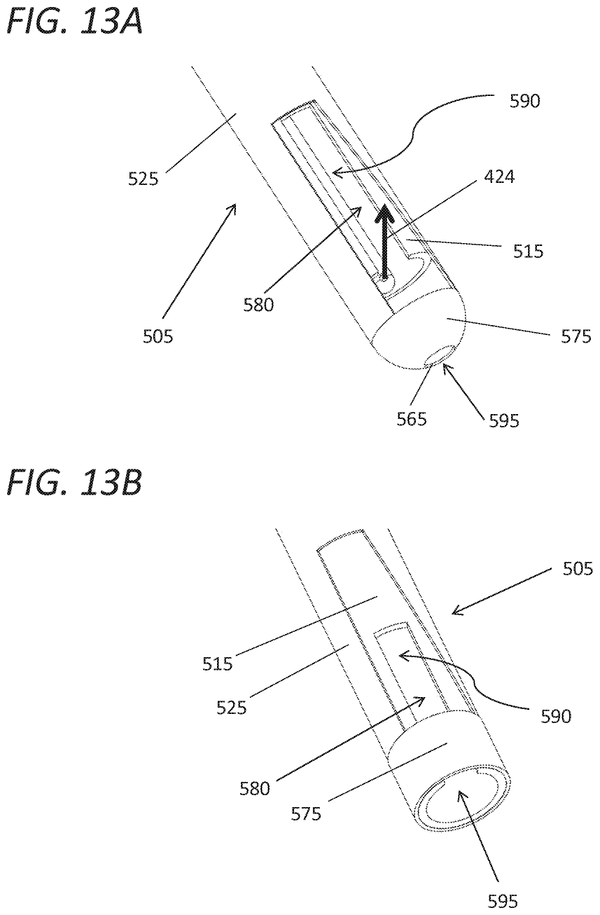

FIG. 13A shows variable aperture tip 505 with elastic tip 575 extending distally from variable aperture sheath 525 such that axial inflow 595 is occluded by axial aperture 565. Aperture 580 is shown fully open and downstream of axial aperture 565; the far-upstream axial aperture 565 experiences only a very slight pressure drop across the aperture and therefore axial inflow 595 is negligible. A large downstream aperture 580 for radial inflow 590 dominates upstream axial inflow 595 through axial aperture 565, rendering the embodiment and configuration of FIG. 13A to substantially exhibit only radial inflow 590. The embodiment and configuration of FIG. 13A illustrate a practical method to simultaneously allow radial inflow 590 and significantly diminish or eliminate axial inflow 595. The configuration of FIG. 13A is appropriate for treating wall-adherent thromboses because impact of direct impingement of radial jet 424 and radial inflow 590 may be intermittently or continuously administered by system controller 180. Aspiration is radial to effectively aspirate thrombotic debris from radial direct impingement cycles, and is also effective to aspirate soft, wall-adherent thrombus employing lower pressures (lower infusion setpoints).

FIG. 13B shows variable aperture tip 505 with elastic tip 575 shown in the retracted position for unoccluded axial inflow 595; hydrodynamic tubing 416 is omitted for clarity. Elastic tip 575 is constructed of a flexible or elastic material (latex, silicone rubber, polyurethane, nylon mesh, etc.) that can be fully retracted over the distal terminus of variable aperture catheter 515. This configuration shows radial inflow 590 through aperture 580 along with unoccluded axial inflow 595 through the distal terminus of the assembly. Variable aperture sheath 525 may also be rotated about variable aperture catheter 515 to close aperture 580 thereby eliminating radial inflow 590.

System controller 180 has setpoint control over variable aperture actuator 599, such that variable aperture catheter 515 is properly configured for any thrombectomy operating mode executed within a thrombectomy procedure. The translational and rotational interaction between variable aperture catheter 515 and variable aperture sheath 525, optionally including elastic tip 575, act as valves to control aspirate inflow in the axial and radial directions. Collectively, FIG. 12A through FIG. 13B present embodiments and configurations whereby external controls are used to coordinate the opening and closing of both axial and radial valves for fluid transfer into or out of a catheter. Different configurations of variable aperture tip 505 are under setpoint control by system controller 180 to provide selectable inflow and outflow in either radial or axial directions. Axial inflow 595 is indicated for vessel-centered thrombi; radial inflow 590 is indicated for wall-adherent thrombi. Variable aperture sheath 525 may also be axially and/or rotationally manipulated with respect to variable aperture catheter 515 by extracorporeal manual input, thus exercising clinician control or override over system controller 180.

During any thrombectomy operating mode, system controller 180 concurrently coordinates the infusion setpoint, the aspiration setpoint and the variable aperture actuator setpoint; system controller 180 thereby controls the net aspiration of fluid flow into and out of the thrombectomy system and, and concomitantly the patient. Clog clearing tip 405 and variable aperture catheter 505 represent embodiments that either contain or release radial jet 424 from impacting intravascular tissue. System controller 180 concomitantly monitors variable aspirate data and aspirate characteristic while periodically updating any or all setpoints. Statistical inference from variable aspirate data determines the aspirate characteristic and subsequently the thrombectomy operating mode employed by system controller 180.

The thrombectomy system of the present invention employs a system controller that orchestrates multiple thrombectomy operating modes during the course of a single procedure by employing a decision tree such as the example flowchart of FIG. 1C. Viable blood is aspirated at a low rate and may be retained for reinfusion; thrombus-laden blood is rapidly aspirated to waste. Clogs are automatically detected and cleared. Wall-adherent thrombus is treated by employing radial direct impingement modes to effect hydrodynamic erosion external to variable aperture tip 505. The thrombectomy system of the present invention thereby provides the clinician with a broader treatment range and the ability to better establish a procedure endpoint that is more favorable for patient outcome.

The differential viscometer of FIG. 2 provides a structure and methodology for the in-process determination of viscous and rheological properties of any liquid in broad variety of applications. Industrial and automotive applications include real-time analyses of the viscous and rheological properties of engine or gearbox oil at any temperature. Manufacturing applications include in-process measurement of viscous and rheological properties of process fluids with analytical instrumentation located in optimum locations including pipes and tanks, as well as infinite reservoirs such as lakes or oceans. Manufacturing batch processes are improved by the ability to rapidly measure the viscosity of a liquid at any location in a tank; the homogeneity of viscosity within a tank or batch may be quantified. Continuous processes are similarly improved by the ability to rapidly measure the viscosity of a liquid flowing through a pipe. Large-scale applications include dredging operations whereby solid debris are rapidly pumped while water is slowly pumped, separation of oil and water in a vessel, etc.

The invention is presented herein in the context of a thrombectomy system, however many inventive subsystems have relevance in other disciplines including: industrial, scientific, automotive, civil engineering, in addition to the medical-device applications presented herein. The invention discloses the methodology to inexpensively redesign or retrofit existing products to incorporate the ability to discriminate inhomogeneous inflow and subsequently update a control output setpoint and optional valve positions. Inhomogeneous inflow may be separated based upon variable data input including viscosity, absorption, conductivity, etc.; valves may be used to divert fluid to different reservoirs. Homogeneous inflow may be periodically characterized for changes in variable input data over time to determine fluid degradation. The scope and detail of this disclosure, combined with a broad array of embodiments enable persons skilled in the art to implement viscometry and associated control systems in ubiquitous applications.

### IDENTIFICATION OF REFERENCE NUMERALS

| | |
|---|---|
| 110 | Water @ 20% |
| 113 | Water @ 100% |
| 115 | H&H @ 40% |
| 120 | Warm SAE 5W20 @ 60% |
| 125 | Cold SAE 5W20 @ 80% |
| 130 | SAE 30 @ 100% |
| 145 | inlet reservoir |
| 155 | discharge reservoir |
| 160 | catheter |
| 165 | pressure transducer |
| 175 | aspirate pump |
| 180 | system controller |
| 215 | inlet pressure data table |
| 221 | viable blood reservoir |
| 225 | pressure-speed graph |
| 226 | waste reservoir |
| 235 | max sensitivity region |
| 240 | max sensitivity domain |
| 242 | sub-range HH |
| 244 | sub-range 5W20@150 |
| 246 | sub-range 5W20@60 |
| 248 | sub-range SAE30 |
| 251 | emitter unit |
| 256 | detector unit |
| 261 | light |
| 271 | clear tubing |
| 280 | photo-detector |
| 285 | aspirate under test |
| 285 | intravascular freestream |
| 291 | upstream aspirate |
| 293 | interface |
| 296 | downstream aspirate |
| 297 | hatch legend |
| 298 | flow direction legend |
| 315 | upstream electrode |
| 325 | downstream electrode |
| 380 | conductivity analyzer |
| 405 | clog clearing tip |
| 416 | hydrodynamic tubing |
| 421 | axial nozzle |
| 423 | radial nozzle |
| 424 | radial jet |
| 425 | tangential nozzle |
| 438 | saline reservoir |
| 450 | analytical instrumentation |
| 475 | infusion pump |
| 505 | variable aperture tip |
| 515 | variable aperture catheter |
| 523 | valve V |
| 525 | valve W |
| 525 | variable aperture sheath |
| 527 | valve F |
| 535 | filter |
| 550 | jet tip distance |
| 565 | axial aperture |
| 575 | elastic tip |
| 580 | aperture |
| 590 | radial inflow |
| 595 | axial inflow |
| 605 | probe viscometer |
| 625 | probe cover |
| 635 | probe inlet tube |
| 645 | probe outlet tube |
| 685 | probe handle |
| 655 | keypad display |
| 675 | probe pump |
| 685 | probe handle |

What is claimed:

1. An apparatus comprising:
a conduit having a first end configured to receive a first fluid;
a controlled source of differential pressure (CSDP) fluidically coupled to the conduit;
one, and only one, sensor fluidically coupled to the conduit between the first end and the CSDP and configured to generate data indicative of pressure within the conduit; and
a controller operatively coupled to the CSDP and the sensor, the controller configured to:
(a) operate the CSDP at a first setpoint to establish a first aspiration condition within the conduit, whereby the first fluid flows through the conduit toward the CSDP;
(b) receive a set of data from the sensor and determine, from the set of data, a first pressure measurement corresponding to the first aspiration condition established by the first setpoint;
(c) determine a first data sub-range of which the first pressure measurement is a member, the first data sub-range being a member of a first set of predetermined data sub-ranges corresponding to the first setpoint;
(d) determine a second setpoint for the CSDP based at least in part upon the first data sub-range; and
(e) operate the CSDP at the second setpoint to establish a second aspiration condition within the conduit.

2. The apparatus of claim 1, wherein the first data sub-range comprises a first alphanumeric descriptor, and wherein the controller is further configured to assign the first alphanumeric descriptor to the first fluid based on the first data sub-range.

3. The apparatus of claim 1, wherein the controller accesses a plurality of sets of data sub-ranges, each set corresponding to a respective setpoint of the CSDP.

4. The apparatus of claim 1, wherein each member of the first set of predetermined data sub-ranges comprises a fixed interval of values.

5. The apparatus of claim 1, wherein the controller accesses the first set of predetermined data sub-ranges from memory.

6. The apparatus of claim 1, wherein the second setpoint comprises an updated setpoint equal to the first setpoint.

7. The apparatus of claim 1, wherein the second setpoint comprises an updated setpoint not equal to the first setpoint.

8. The apparatus of claim 7, wherein the second aspiration condition is such that the first fluid flows through the conduit away from the CSDP.

9. The apparatus of claim 1, wherein the controller is further configured to operate the CSDP according to an operating mode, the operating mode comprising:
(a) a setpoint of the CSDP; and
(b) a continuation parameter defining an extent of operation of the CSDP at the setpoint, the continuation parameter comprising at least one of:
(i) a duration of operation, and
(ii) a threshold condition.

10. The apparatus of claim 9, further comprising at least one of:
(a) a valve fluidically coupled to the conduit;
(b) a liquid pump in fluid communication with the conduit; and
(c) an actuator coupled to the apparatus, wherein:
the controller is further operatively coupled to each of the valve, the liquid pump, and the actuator that is present in the apparatus, and wherein the operating mode further comprises a respective setpoint for each of the valve, the liquid pump, and the actuator that is present in the apparatus.

11. The apparatus of claim 9, wherein the threshold condition comprises a statistical condition derived from the set of data.

12. The apparatus of claim 9, wherein the controller is further configured to update the operating mode based at least in part upon the first data sub-range.

13. The apparatus of claim 9, wherein the controller is further configured to select an operating mode from a set of operating modes based at least upon the first data sub-range.

14. The apparatus of claim 12, wherein the CSDP comprises a pump having a rotatable shaft, a cycle corresponds to a rotational displacement of the shaft; and the controller is further configured to update the operating mode on a cycle-to-cycle basis.

15. The apparatus of claim 13, wherein the controller is further configured to:
(a) compare the first data sub-range to a data sub-range determined during a previous operation of the apparatus at the first setpoint;
(b) update the operating mode to the same operating mode when the first data sub-range is the same as the data sub-range determined during the previous operation of the apparatus; and
(c) update the operating mode to a different operating mode when the same operating mode has been repeated for a threshold number of operations.

16. The apparatus of claim 2, wherein the controller is further configured to provide an audible indication corresponding to the first alphanumeric descriptor, wherein the audible indication comprises a pitch level or a volume level selected according to the first alphanumeric descriptor.

17. The apparatus of claim 2, wherein the first alphanumeric descriptor corresponds to one or more of: water, saline, oil, blood, thrombus, clot, clog, half and half, SAE 30 motor oil, 5W20 motor oil, or 0W40 motor oil, and mixtures thereof in any proportion.

18. A method of determining a sub-range of pressure values for operation of an aspiration apparatus, the method comprising:
(a) establishing a first setpoint of a controlled source of differential pressure (CSDP);
(b) establishing a first range of pressure values and a first alphanumeric descriptor corresponding to the first setpoint;
(c) receiving a first fluid into a conduit, the first fluid corresponding to the first alphanumeric descriptor;
(d) operating the CSDP at the first setpoint to establish a first aspiration condition whereby the first fluid flows through the conduit toward the CSDP;
(e) receiving a set of data from a sensor, the set of data being indicative of pressure within the conduit; and
(f) determining the sub-range of pressure values corresponding to the first setpoint and the first alphanumeric descriptor based at least in part upon: (i) the set of data, and (ii) the first range of pressure values.

19. The method of claim 18, wherein the first range of pressure values comprises an arbitrary range.

20. The method of claim 18, wherein establishing the first range of pressure values and the first alphanumeric descriptor comprises retrieving at least one of: (i) the first range of pressure values, and (ii) the first alphanumeric descriptor, from memory.

21. The method of claim 18, further comprising storing the sub-range of pressure values in memory.

22. The method of claim 18, wherein the first alphanumeric descriptor is selected from a set of alphanumeric descriptors stored in memory.
